(19) United States
(12) Patent Application Publication
(10) Pub. No.: US 2025/0019766 A1
(43) Pub. Date: Jan. 16, 2025

(54) TUMOR CELL IDENTIFICATION BY MAPPING MUTATIONS IN BULK DNA SEQUENCES TO SINGLE CELL RNA SEQUENCES

(71) Applicant: AMAZON TECHNOLOGIES, INC., RENO, NV (US)

(72) Inventors: Samuel Anthony DANZIGER, Seattle, WA (US); Haibao TANG, Cupertino, CA (US); David HECKERMAN, Bellevue, WA (US); Frank Wilhelm SCHMITZ, Seattle, WA (US); Alena HARLEY, Los Gatos, CA (US)

(21) Appl. No.: 18/279,714

(22) PCT Filed: Aug. 2, 2023
(86) PCT No.: PCT/US2023/071519

§ 371 Date: Aug. 31, 2023

Related U.S. Application Data
(60) Provisional application No. 63/397,597, filed on Aug. 12, 2022.

(51) Int. Cl. C12Q 1/6886; C12Q 1/6881

(52) U.S. Cl. CPC C12Q 1/6886; C12Q 1/6881; C12Q 2600/106; C12Q 2600/156

(57) ABSTRACT

Disclosed herein are methods for classifying a cell present in a sample. such as a tumor, of a subject. comprising: sequencing bulk DNA from first and second (e.g., tumor and healthy) a subject's tissue samples; classifying somatic variants as first or second sample alleles; sequencing RNA from the cell; aligning each RNA sequence with the bulk DNA; classifying each RNA sequence as a first, a second, or an unknown allele sequence, depending on whether it substantially aligns with the first, the second sample allele, or cannot be determined; and identifying the cell as a first, a second, or an unknown cell, based on the classifying of each of the plurality of RNA sequences. Methods can comprise validating identification by allelic frequency of germ-line variants in the RNA sequences. The methods provide improved characterization of heterogenous cell populations, such as cell populations contaminated with cells from different sources, or tumor populations.

### CROSS-REFERENCE TO RELATED APPLICATIONS

The present application claims the benefit of U.S. Provisional Application No. 63/397,597 filed on Aug. 12, 2022, the entire contents of which are incorporated herein by reference.

### 1. BACKGROUND

Whole genome sequencing involves sequencing the full DNA of cells of a tissue of interest. A similar technique, exome sequencing, involves sequencing the full complement of exons in the cells of the tissue of interest. The two techniques may generically be referred to as WxS sequencing. WxS sequencing is performed on bulk tissue, meaning that the genomic or exomic information of all the cells in the tissue is pooled prior to sequencing. Accordingly, genomic or exomic variation between cells of the tissue cannot be resolved by WxS sequencing.

One application of WxS sequencing is in cancer diagnostics. WxS sequencing can be performed on a tumor sample from a subject and from a healthy (non-cancerous) tissue sample from the subject. The two sequences can be compared and cancer-specific mutations. such as driver mutations that caused tumor cells to become cancerous or subclonal mutations that can endow tumor cells with the ability to survive therapy and lead to relapse, can be identified.

As stated above, WxS sequencing cannot resolve variation between cells of a tissue. Because tumors contain a variety of cells, including cancer cells (which can further be members of divergent subclonal lineages), stromal cells, non-cancerous cells, and immune cells, WxS sequencing may fail to provide information that would be useful to the clinician attempting to treat the subject's cancer.

Accordingly, it would be desirable to have increased resolution of genetic variation between cells of a tissue, such as between cells of a tumor.

### 2. SUMMARY

This disclosure relates to a method for classifying a cell present in a first sample from a subject. The method comprises sequencing bulk DNA from a first sample from the subject. In some embodiments, the first sample can be a tumor sample, i.e., the first sample can be from a tumor.

The method also comprises sequencing bulk DNA from a second sample from the subject. In some embodiments, the second sample can be a normal or healthy tissue sample, i.e., the second sample can be from healthy tissue.

In some embodiments, the sequencing bulk DNA from the first sample or the sequencing bulk DNA from the second sample can comprise whole genome sequencing.

In some embodiments, the sequencing bulk DNA from the first sample or the sequencing bulk DNA from the second sample can comprise exome sequencing.

The method also comprises classifying each somatic variant between the first sample bulk DNA sequence and the second sample bulk DNA sequence as a first sample allele if present in the first sample bulk DNA sequence or a second sample allele if present in the second sample bulk DNA sequence. In some embodiments, the first sample allele can be a tumor allele and the second sample allele can be a normal allele.

The method also comprises sequencing RNA from the cell, to yield a plurality of cell RNA sequences. In some embodiments, the sequencing RNA from the cell yields a plurality of cell RNA sequences each comprising a unique molecular identifier (UMI) and about 100 nucleotides from the 3′ end of an RNA present in the cell.

Also, the method comprises aligning each cell RNA sequence of the plurality of cell RNA sequences with the first sample bulk DNA sequence and the second sample bulk DNA sequence.

In addition, the method comprises classifying each cell RNA sequence of the plurality of cell RNA sequences as a second allele sequence if the cell RNA sequence substantially aligns with a second sample allele from the second sample bulk DNA sequence, as a first allele sequence if the cell RNA sequence substantially aligns with a first sample allele from the first sample bulk DNA sequence, or as an unknown allele sequence if the cell RNA sequence does not substantially align with either the second sample bulk DNA sequence or the first sample bulk DNA sequence. In some embodiments, the first allele sequence can be a tumor allele sequence and the second allele sequence can be a normal allele sequence.

In some embodiments, the method can further comprise determining a sequence-specific error rate for each cell RNA sequence of the plurality of RNA sequences: wherein the classifying each cell RNA sequence is based in part on the sequence-specific error rate.

The method can also comprise identifying the cell as a first cell, a second cell, or an unknown cell, based at least in part on the classifying of each cell RNA sequence of the plurality of cell RNA sequences. In some embodiments. the first cell can be a tumor cell and the second cell can be a healthy cell.

In some embodiments, the method can further comprise determining a general error rate for the sequencing RNA from the cell: wherein the classifying each cell RNA sequence of the plurality of RNA sequences is based in part on the general error rate or the identifying the cell is further based in part on the general error rate.

In some embodiments, the identifying the cell can comprise a Bayesian analysis of a number of first allele sequences and a number of second allele sequences.

In some embodiments, the first sample is a tumor sample and the second sample is a healthy tissue sample. In some embodiments, the first cell is a tumor cell. In some embodiments, the method further comprises determining a subclone status of the tumor cell.

In some embodiments, the method can further comprise generating a subclone peptide that is at least in part encoded by a cell RNA sequence from the tumor cell and specific for the subclone status of the tumor cell: and formulating an immunogenic composition comprising the subclone peptide.

In some embodiments, the method can further comprise generating a non-subclone peptide, wherein the non-subclone peptide is derived from a cell that has a different subclone status than the tumor cell: and including the non-subclone peptide in the immunogenic composition. In some embodiments, the cell that has a different subclone status than the tumor cell is from the tumor of the subject.

In some embodiments, the method can further comprise administering the immunogenic composition to the subject. In some embodiments, the administering can be performed prior to or simultaneously with delivering one or more other therapeutic agents for the tumor to the subject. Alternatively or in addition, one or more of the generating the subclone peptide, the formulating, the generating the non-subclone peptide, the including, and the administering can be performed after delivering one or more other therapeutic agents and/or other immunogenic compositions to the subject.

In some embodiments, the method can further comprise determining the mutational history of the tumor cell.

In some embodiments, the method can further comprise the step of validating the step of identifying the cell as a first cell, second cell, or an unknown cell, based at least in part on an allelic frequency of germ-line variants in the cell RNA sequences. The method can comprise the step of identifying germ-line variants in a first and a second sample nucleic acid sequences (e.g., bulk DNA sequences, RNA sequences, cDNA sequences) and determining a copy number at each sequence region comprising each germ-line variant in the first sample nucleic acid sequence and the second sample nucleic acid sequence. The method can comprise selecting one or more determinative germ-line variants (DGLVs) from the germ-line variants in the first and second samples with a first B-allele frequency from the first sample nucleic acid sequence and a second B-allele frequency from the second sample nucleic acid sequence, wherein the first and second B-allele frequencies are statistically different. The sequence region comprising each DGLV can have a ratio of the copy number in the second sample nucleic acid sequence to copy number in the first sample nucleic acid sequence. In some embodiments, the ratio of copy numbers is about 2:3, about 1:2. about 2:5. about 1:3, about 2:7, about 1:4, about 2:9, or about 1:5. In some embodiments the ratio of copy numbers is about 2:1. In some embodiments, the ratio of copy numbers is about 1:1. The method can further comprise aligning each cell RNA sequence of the plurality of cell RNA sequences with each of the DGLVs and determining a B-allele frequency of each DGLV in the plurality of cell RNA sequences. The method can further comprise validating the step of identifying the cell as a first cell, a second cell, or an unknown cell, based at least in part on the B-allele frequency of each DGLV in the plurality of cell RNA sequences.

The germ-line variant can be any type of mutation. In some embodiments, the germ-line variant is a mutation selected from the group consisting of a single nucleotide polymorphism, an insertion, a deletion, a translocation, and combinations thereof.

The statistical difference between first and second B-allele frequencies can be any statistical difference. In some embodiments, the statistical difference is p<0.050. The statistical difference can be determined by any statistical test. In some embodiments. the statistical difference is determined by a test selected from the group consisting of binomial test, Kruskal-Wallis one-way analysis of variance. Mann-Whitney U test, Siegel-Tukey test, student's T test, Tukey's range test, and combinations and hybrids thereof. The second B-allele frequency can be not statistically different from any value as determined by a second statistical test. In some embodiments, the second B-allele frequency is not statistically different from 0.50 as determined by a second statistical test. The first B-allele frequency can be statistically different from any value as determined by a first statistical test. In some embodiments, the first B-allele frequency is statistically different from 0.50 as determined by a first statistical test. The first and second statistical test can be any type of statistical test with any p value. In some embodiments. the first statistical test and/or the second statistical test can be a binomial test with p<0.050.

The B-allele frequency of a DGLV in the cell nucleic acid (e.g., RNA) sequences validating the step of identifying the cell as a second cell can be of any range. In some embodiments, the B-allele frequency of the DGLV in the plurality of cell RNA sequences validating the step of identifying the cell as a second cell ranges from about 0.40 to about 0.50.

The B-allele frequency of a DGLV in cell sequence nucleic acid (e.g., RNA) sequences validating a first cell can be of any range. In some embodiments, the B-allele frequency of the DGLV in the plurality of cell RNA sequences validating the step of identifying the cell as a first cell ranges from about 0.00 to about 0.32.

### 3. BRIEF DESCRIPTION OF THE DRAWINGS

FIG. 1 presents hypothetical mappings of WxS sequencing data to scRNA sequencing data to illustrate principles used in methods of the present disclosure. FIG. 1 discloses SEQ ID NOS: 1, 2, 1, and 3-9, respectively, in order of appearance.

FIG. 2 presents hypothetical allele classification and cell identification to illustrate principles used in methods of the present disclosure.

FIG. 3 graphs tumor probability determined by the methods described herein for cells of various types as determined by gene expression profiling, as described in Example 3.

FIGS. 4A and 4B are graphs showing the copy number across the genome for BC362 cancer cells (cells obtained from a patient biopsy) and B-allele frequency for single cell RNA sequencing (scRNAseq) reads across the genome for non-cancer and cancer cells (BC362 biopsy cells). FIG. 4A shows the genome position (separated by chromosome (x-axis)) versus the copy number (y-axis). The graph indicates the type of copy number variation (e.g., duplication (copy number>2, black): duplication with loss of heterozygosity (copy number=2, gray); region of no copy number change and no loss of heterozygosity (reference, copy number=2, gray): copy number neutral loss of heterozygosity (copy number=2, black): deletion (copy number=1, black). FIG. 4B shows B-allele frequency (y-axis) versus genomic position (separated by chromosome (x-axis)) for scRNAseq reads. Reads from cancer cells are shown in black, reads from non-cancer cells are shown in gray. B-allele frequencies in cancer cells in sequence regions having neither copy number change, nor loss of heterozygosity. are not shown. The graph reveals that most non-cancer cells have a B-allele frequency of about 0.5, while most cancer cells have a B-allele frequency of less than about 0.5. Sequence regions of duplication with loss of heterozygosity and/or deletion have a B-allele frequency of about 0. Sequencing regions of increasing duplication have a decreasing B-allele frequency.

FIGS. 5A and 5B are graphs showing the copy number across the genome for BH956 cancer cells (cells obtained from a patient biopsy) and B-allele frequency for single cell RNA sequencing (scRNAseq) reads across the genome for non-cancer and cancer cells (BH956 biopsy cells). FIG. 5A shows the genome position (separated by chromosome (x-axis)) versus the copy number (y-axis) in BH956 cells. The graph indicates the type of copy number variation (e.g., duplication (copy number>2, black): duplication with loss of heterozygosity (copy number=2, gray): region of no copy number change and no loss of heterozygosity (reference, copy number=2. gray); copy number neutral loss of heterozygosity (copy number=2, black): deletion (copy number=1, black). FIG. 5B shows B-allele frequency (y-axis) versus genomic position (separated by chromosome (x-axis)) for scRNAseq reads. B-allele frequencies in sequence regions of cancer cells having neither copy number change, nor loss of heterozygosity, are not shown. The graph reveals that most non-cancer cells have a B-allele frequency of about 0.5, while most cancer cells have a B-allele frequency less than about 0.5. Sequence regions of duplication with loss of heterozygosity and deletion have a B-allele frequency of about 0. Sequence regions of increasing duplication have a decreasing B-allele frequency.

FIG. 6 is a graph of receiver operating characteristic (ROC) curves of the false positive rate (x-axis) versus true positive rate (y-axis) for methods of identifying cells as cancer or non-cancer cells (e.g., tumor or healthy cells). The curves for the methods of identifying cells based on 1) only somatic mutations (black, area=0.9), 2) only B-allele frequency (gray, area=0.98), and 3) a combination of somatic mutations and B-allele frequency (light gray, area=0.985) are shown. The graph shows that methods of cell identification based on either somatic mutations or B-allele frequency can identify cells as true positives with a greater probability than false positives (e.g., greater probability of detection than false alarm), however the methods can be further improved by accounting for both somatic mutations and B-allele frequency.

FIG. 7 is a graph of violin plots of different cell types (x-axis) from patients versus the probability a cell is a tumor cell. The method used to identify cells as healthy (e.g., non-cancerous) or tumor cells was based on both somatic mutations and B-allele frequency of germ-line variants. The graph shows an increased probability of identifying cancer cells and decreased probability of identifying healthy cells as cancer cells compared to the methods relying on solely somatic mutations (e.g., FIG. 3). Cell types are inferred based on transcriptomic profiles of the cells, which include (from left to right) naïve B-cells, basal-like breast cancer (BLBC), hepatic stellate cells, Her2 (human epidermal growth factor receptor 2) enriched breast cancer (HER2E), Luminal-like A (LumA) breast cancer, natural killer (NK) cells, adipocytes, microvascular (mv) derived endothelial cells, macrophages, Luminal-like B (LumB) breast cancer, CD4+ T effector memory (Tem) cells, fibroblasts, regulatory T (Treg) cells, CD8+ T central memory (Tcm) cells, endothelial cells, cycling perivascular-like cancer associated cells, monocytes, plasma cells, cells that could not be classified (e.g., ‘unknown’), CD4+ T central memory (Tcm) cells, CD8+ T cells, CD4+ T cells, CD8+ T effector memory (Tem) cells, melanocytes (e.g., healthy skin cells). The high variability of tumor cell gene expression makes them difficult to classify: a breast cancer cell may appear to be a healthy melanocyte. Methods described herein can improve the classification of cells as healthy cells or tumor cells.

FIG. 8 is a graph of single cell RNAseq data clustering for multiple cell types of patient origin based on the clustering of transcriptomic profiles of each cell. Multiple cell types were analyzed by single cell RNAseq data for somatic mutations and B-allele frequency (BAF) of germ-line variants, followed by assignment of a probability that the cell was a cancer cell. The probability of a cell being a cancer cell (e.g., tumor cell) is displayed as a gradient (1.0=100% probability the cell is a cancer cell, black; 0.0=0% probability the cell is a cancer cell, light gray). Clusters of cells are labeled by cell type (e.g., dendritic cell (DC)). The graph shows a high probability of cells being cancer cells (e.g., true positive) when both somatic mutations and B-allele frequency are used to generate the cancer cell probability. These results are from the same experiments which are summarized in the FIG. 7 violin plot.

FIG. 9 is a graph of single cell RNAseq data for multiple cell types based on the clustering of transcriptomic profiles of each cell. Multiple cell types were analyzed by single cell RNAseq data for somatic mutations (and not B-allele frequency of germ-line variants) and assigned a probability that the cell is a cancer cell. The probability a cell being a cancer cell (e.g., tumor cell) is displayed as a gradient (1.0=100% probability the cell is a cancer cell, black; 0.0=0% probability the cell is a cancer cell, light gray). Clusters of cells are labeled by cell type (e.g., B cell, myeloid cell). These results are from the same experiments which are summarized in the FIG. 3 violin plot.

FIG. 10 is a graph of single cell RNAseq data for multiple cell types. Multiple cell types were analyzed by single cell RNAseq data for somatic mutations and B-allele frequency of germ-line variants, followed by assignment of a probability that the cell is a cancer cell. The probability of a cell being a cancer cell (e.g., tumor cell) is displayed as a gradient (1.0=100% probability the cell is a cancer cell, black: 0.0=0% probability the cell is a cancer cell, light gray). Clusters of cells are labeled by cell type (e.g., B cell, myeloid cell). The graph shows that the melanoma cells have a higher probability (e.g., true positive) when both somatic mutations and B-allele frequency are used to generate the cancer cell probability, as compared to somatic mutations alone as shown in FIG. 9. These results are from the same experiments which are summarized in the violin plot graph of FIG. 11.

FIG. 11 is a violin plot of different cell types (x-axis) versus the probability a cell is a tumor cell. The method used to identify cells as healthy (e.g., non-cancerous) or tumor cells was based on both somatic mutations (as described in Example 3) and B-allele frequency of germ-line variants. The graph shows that adding B-allele frequency of germ-line variants to identification by somatic mutations (e.g., in comparison to the results in FIG. 3, identification by only somatic mutations) achieves a higher probability of correctly identifying cancer cells (e.g., melanoma, identifying true positives), while reducing the probability of identifying non-cancer cells as cancer cells (e.g., decrease false positives). Cell types include (from left to right) myeloid cells, natural killer (NK) or T cells, melanoma (cancer cells), erythrocytes, fibroblasts, B cells, and granulocytes.

### 4. DETAILED DESCRIPTION

This disclosure relates to methods in which genomic or exomic variants found by WxS sequencing can be mapped to cell-specific sequence information found by single cell RNA (scRNA) sequencing. In some embodiments, by aligning each cell-specific sequence found by scRNA sequencing of first tissue to corresponding sequences from WxS sequencing of first tissue and second tissue, each cell-specific sequence can be classified as a first allele sequence, a second allele sequence, or an unknown allele sequence, and from the cell-specific sequences of each cell, the cell can be identified as a first cell or a second cell. Identified cells can be further validated as first cells, second cells, or unknown cells based, at least in part, on allelic frequency (e.g., a B-allele frequency) of germ-line variants in the cell RNA sequences.

In some embodiments, the first sample can be from a first subject and the second sample can be from a second subject. If the cell is from the first sample and the first sample is suspected of being contaminated by cells from the second subject, the method can be used to identify which subject's sample is the source of the cell, i.e., whether the cell is a first cell from the first subject or a second cell from the second subject. Performed over multiple cells, a probability of contamination of the first sample can be established.

In some embodiments, the first sample can be from a tumor of a subject and the second sample can be from a normal or healthy tissue of the subject. The method can be used to analyze cell heterogeneity of the subject's tumor, among other purposes.

Given the great interest in diagnosing, monitoring. and treating cancer, the description will generally refer to tumor and healthy samples, alleles, sequences, and cells. It should be borne in mind that the description is generally applicable to identifying cells in any heterogenous cell population, in situations where members of the heterogenous cell population have allele sequences attributable to a first or a second sample.

Even further resolution is possible, to the level of subclone or mutational lineage of tumor cells, or cell type of healthy cells. In other words, the methods disclosed herein can provide for increased resolution of genetic variation between cells of a tissue, such as between cells of a tumor.

All publications and patents cited in this disclosure are incorporated by reference in their entirety. To the extent, the material incorporated by reference contradicts or is inconsistent with this specification, the specification will supersede any such material. The citation of any references herein is not an admission that such references are prior art to the present disclosure. When a range of values is expressed, it includes embodiments using any particular value within the range. Further, reference to values stated in ranges includes each and every value within that range. All ranges are inclusive of their endpoints and combinable. When values are expressed as approximations, by use of the antecedent “about.” it will be understood that the particular value forms another embodiment. Reference to a particular numerical value includes at least that particular value, unless the context clearly dictates otherwise. The use of “or” will mean “and/or” unless the specific context of its use dictates otherwise.

Various terms relating to aspects of the description are used throughout the specification and claims. Such terms are to be given their ordinary meaning in the art unless otherwise indicated. Other specifically defined terms are to be construed in a manner consistent with the definitions provided herein. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press. Cold Spring Harbor. NY. As appropriate, procedures involving the use of commercially available kits and reagents are generally carried out in accordance with manufacturer-defined protocols and conditions unless otherwise noted.

As used herein, the singular forms “a,” “an,” and “the” include plural forms unless the context clearly indicates otherwise. The terms “include,” “such as,” and the like are intended to convey inclusion without limitation, unless otherwise specifically indicated.

Unless otherwise indicated, the terms “at least,” “less than,” and “about,” or similar terms preceding a series of elements or a range are to be understood to refer to every element in the series or range. Those skilled in the art will recognize, or be able to ascertain using no more than routine experimentation, many equivalents to the specific embodiments of the invention described herein. Such equivalents are intended to be encompassed by the following claims.

The term “cancer” refers to the physiological condition in subjects in which a population of cells is characterized by uncontrolled proliferation, immortality, metastatic potential, rapid growth and proliferation rate and/or certain morphological features. Often cancers can be in the form of a tumor or mass, but may exist alone within the subject, or may circulate in the blood stream as independent cells, such a leukemic or lymphoma cells. The term cancer includes all types of cancers and metastases, including hematological malignancy, solid tumors, sarcomas, carcinomas and other solid and non-solid tumors. Examples of cancers include, but are not limited to, carcinoma, lymphoma, blastoma, sarcoma, and leukemia. More particular examples of such cancers include squamous cell cancer, small cell lung cancer, non-small cell lung cancer, adenocarcinoma of the lung, squamous carcinoma of the lung, cancer of the peritoneum, hepatocellular cancer. gastrointestinal cancer, pancreatic cancer, glioblastoma, cervical cancer, ovarian cancer, liver cancer, bladder cancer, hepatoma, breast cancer (e.g., triple negative breast cancer, Hormone receptor positive breast cancer), osteosarcoma, melanoma, colon cancer, colorectal cancer, endometrial (e.g., serous) or uterine cancer, salivary gland carcinoma, kidney cancer, liver cancer, prostate cancer, vulvar cancer, thyroid cancer, hepatic carcinoma, and various types of head and neck cancers.

The term “subject” as used herein refers to any animal, such as any mammal, including but not limited to, humans, non-human primates, rodents, mammals commonly kept as pets (e.g., dogs and cats, among others), livestock (e.g., cattle, sheep, goats, pigs, horses, and camels, among others) and the like. In some embodiments, the mammal is a mouse. In some embodiments, the mammal is a human.

The term “tumor cell” as used herein refers to any cell that is a cancer cell or is derived from a cancer cell. The term “tumor cell” can also refer to a cell that exhibits cancer-like properties, e.g., uncontrollable reproduction, resistance to anti-growth signals, ability to metastasize, and loss of ability to undergo programed cell death.

Additional description of the methods and guidance for the practice of the methods are provided herein.

### A. WxS Sequencing

The methods disclosed herein can comprise sequencing bulk DNA from a tumor sample from the subject. The methods can also comprise sequencing bulk DNA from a healthy tissue sample from the subject.

Bulk DNA is used herein to refer to DNA pooled from a plurality of cells within the sample or generated from other nucleic acids pooled from a plurality of cells within the sample. In some embodiments, in whole genome sequencing, genomic DNA can be extracted from all the cells of the plurality and sequenced directly. In exome sequencing, genomic DNA can be extracted from all the cells of the plurality and sheared to fragments, followed by hybridizing fragments containing exons to an array containing corresponding oligonucleotides, and sequencing of the hybridized fragments. Particular techniques for extracting genomic DNA, processing the extracted DNA, and sequencing DNA are well-known and need not be described in detail.

Accordingly, in embodiments, the sequencing bulk DNA from the tumor sample, the sequencing bulk DNA from the healthy tissue sample, or both can comprise whole genome sequencing. Additionally or alternatively, in embodiments, the sequencing bulk DNA from the tumor sample, the sequencing bulk DNA from the healthy tissue sample, or both can comprise exome sequencing.

Additional or alternative techniques for sequencing bulk DNA can be selected by a person of ordinary skill in the art having the benefit of the present disclosure, with it being understood that the sequencing of the bulk DNA should provide sequence information relating to at least some transcribed regions of the genome, for reasons to be discussed herein.

Whether the bulk DNA is genomic, exomic, or another tranche of genomic DNA, sequencing the bulk DNA yields sequence information relating to DNA found in the genomes of cells within the sample. Although bulk DNA sequencing cannot resolve somatic variants to the level of individual cells, in at least some circumstances, it can detect variations in the DNA pool.

Also, though not every cell in a tumor sample is necessarily a tumor cell, a tumor sample will contain tumor cells. The tumor cells are expected to provide one or more somatic variants relative to healthy, non-cancerous cells from nearby healthy tissue.

Accordingly, the methods can also comprise classifying each somatic variant between the tumor sample bulk DNA sequence and the healthy tissue sample bulk DNA sequence. Specifically, each somatic variant can be classified as a tumor allele if present in the tumor sample bulk DNA sequence or a normal allele if present in the healthy tissue sample bulk DNA sequence.

To aid in visualization. consider the four hypothetical sequences depicted in the upper portion of FIG. 1. Three sequences are from a healthy tissue sample bulk DNA sequence (Normal) and one is from a tumor sample bulk DNA sequence (Tumor). The three somatic variants found only in the tumor sequence can be classified as tumor alleles; the one somatic variant found in the second healthy sequence (relative to the other two healthy sequences) can be classified as a normal allele.

### B. scRNA Sequencing

The methods disclosed herein can comprise sequencing RNA from the cell, to yield a plurality of cell RNA sequences. In embodiments, the methods can comprise single cell RNA (scRNA) sequencing.

Generally, scRNA sequencing involves the separation of individual cells from a sample, the generation of cDNA molecules complementary to cellular mRNA and labeled with a cell-specific identifier, a unique nucleotide sequence sometimes called a barcode, which is specific for one and only one source cell, and a unique molecular identifier (UMI), which is specific for an individual cDNA molecule. Accordingly, scRNA sequencing can resolve at least some variation between cells of a tissue.

Generally, the cDNA molecules will comprise the UMI and about 100 nucleotides from the 3′ end of the mRNA. Given the relatively small amount of a single cell's mRNA in comparison to the larger amount of genomic or exomic DNA from a bulk sample comprising a very large number of cells, amplification of the cDNA molecules is generally performed.

Although the cDNA molecules each comprise only about 100 nucleotides from the 3′ end of the mRNA, a gene of the cell can give rise to multiple mRNAs through alternative splicing, alternative polyadenylation, or various other processes. Variations in the reverse transcriptase process can yield cDNAs complementary to different subsequences of identical mRNAs. These phenomena can give rise to scRNA sequencing reads providing overlapping coverage of mRNAs transcribed from a single DNA coding region.

Hypothetical examples of overlapping scRNA sequencing reads are given in the lower portion of FIG. 1. A set of three unique cell RNA sequences labeled as UMI_1, UMI_2, and UMI_3 overlap as shown, indicating derivation from a single allele, Allele #1. Another set of three unique cell RNA sequences labeled as UMI_4, UMI_5, and UMI_6 overlap as shown, indicating derivation from another single allele, Allele #2.

Another aspect of scRNA sequencing to be taken into consideration are error rates. In any sequencing workflow, errors can arise from inadvertent modification of nucleic acid molecules during preparation of samples for sequencing, i.e., from misfunction of reverse transcriptase when preparing cDNAs from mRNA in scRNA sequencing, from misfunction of polymerase when amplifying DNA through PCR or related techniques, etc. Errors can also arise from misreading of nucleic acid molecules during the sequencing process itself. From either origin of errors, certain subsequences can be more prone to sequencing errors than others, which can give target sequences specific error rates.

Accordingly, any sequencing workflow has a general error rate, i.e., some probability that any one nucleotide will be missed, misidentified, etc. regardless of the target sequence. General or background error rates for scRNA sequencing are commonly in the range of 0.01 to 0.0001 (phred quality scores of 20-40), representing one error in every 1,00 to 10,000 bases. The general error rate is also referred to herein as a background error rate. Also, for any given workflow, any target sequence can have a sequence-specific error rate. The sequence-specific error rate is also referred to herein as a contextual error rate.

Given the smaller amount of source nucleic acids in scRNA sequencing in contrast to WxS sequencing, errors can be of greater import to the analysis of scRNA sequences than to that of genomic/exomic sequences taken from bulk tissue.

The methods can further comprise providing a general error rate for the sequencing RNA from the cell. Additionally or alternatively, the methods can further comprise providing a sequence-specific error rate for sequencing each cell RNA sequence.

Error rates can be estimated by use of known techniques in the art and need not be described in detail.

### C. Aligning Cell RNA Sequences with Bulk DNA Sequences

After sequencing of the tumor sample bulk DNA, the healthy tissue sample bulk DNA, and the RNA of the cell, the method can comprise aligning each cell RNA sequence with the tumor sample bulk DNA sequence and the healthy tissue sample bulk DNA sequence.

Sequence alignment is a well-known technique in bioinformatics and can be performed by any suitable method. However, for determining the degree of alignment between sequences, computer programs that make multiple alignments of sequences can be useful. for example Clustal W (Thompson. Higgins. Gibson. Nucleic Acids Res., 22:4673-4680, 1994). If desired, the Clustal W algorithm can be used together with BLOSUM 62scoring matrix (Henikoff and Henikoff, Proc. Natl. Acad. Sci. USA, 89:10915-10919, 1992) and a gap opening penalty of 10 and gap extension penalty of 0.1, so that the highest order match is obtained between two sequences wherein at least 50% of the total length of one of the sequences is involved in the alignment. Other methods that can be used to align sequences are the alignment method of Needleman and Wunsch (Needleman and Wunsch, J. Mol. Biol., 48:443, 1970) as revised by Smith and Waterman (Smith and Waterman, Adv. Appl. Math., 2:482, 1981) so that the highest order match is obtained between the two sequences and the number of identical amino acids is determined between the two sequences. Other methods to calculate the percentage identity between two amino acid sequences are generally art recognized and include, for example, those described by Carillo and Lipton (Carillo and Lipton, SIAM J. Applied Math., 48:1073, 1988) those described in Computational Molecular Biology, Lesk, Ed., Oxford University Press, New York, 1988, Biocomputing: Informatics and Genomics Projects.

Generally, computer programs will be employed for such calculations. Programs that compare and align pairs of sequences, like ALIGN (Myers and Miller, CABIOS, 4:1 1-17, 1988), FASTA (Pearson and Lipman, Proc. Natl. Acad. Sci. USA, 85:2444-2448, 1988: Pearson, Methods in Enzymology, 183:63-98, 1990) and gapped BLAST (Altschul et al., Nucleic Acids Res., 25:3389-3402. 1997), BLASTP, BLASTN, or GCG (Devereux, Haeberli, Smithies, Nucleic Acids Res., 12:387, 1984) can also be useful for this purpose.

### D. Classifying Cell RNA Sequences as Normal, Tumor, or Unknown

However the alignment of each cell RNA sequence with the tumor sample bulk DNA sequence and the healthy tissue sample bulk DNA sequence is performed, there are three possible outcomes.

1. The cell RNA sequence substantially aligns with a normal allele from the healthy tissue sample bulk DNA sequence.

2. The cell RNA sequence substantially aligns with a tumor allele from the tumor sample bulk DNA sequence.

3. The cell RNA sequence does not substantially align with either a healthy tissue sample bulk DNA sequence or a tumor sample bulk DNA sequence.

In the first outcome, the cell RNA sequence can be classified as a normal allele sequence.

In the second outcome, the cell RNA sequence can be classified as a tumor allele sequence.

In the third outcome, the cell RNA sequence can be classified as an unknown allele sequence.

Continuing the hypothetical example shown in FIG. 1, UMI_1, UMI_2, and UMI_3 found by scRNA sequencing each align with a tumor allele from the tumor sample bulk DNA sequence found by WxS sequencing. Accordingly, the cell RNA sequences of UMI_1, UMI_2, and UMI_3 can be classified as tumor allele sequences. Similarly, UMI_4. UMI_5, and UMI_6, also found by scRNA sequencing. each align with a normal allele from the healthy tissue sample bulk DNA sequence found by WxS sequencing. UMI_4, UMI_5, and UMI_6 can be classified as normal allele sequences.

In some embodiments, the classifying the cell RNA sequence can be based in part on the general or background error rate of the RNA sequencing.

Additionally or alternatively, in some embodiments, the classifying the cell RNA sequence can be based in part on the sequence-specific or contextual error rate of the RNA sequencing.

### E. Identifying Cells as Tumor, Healthy, or Unknown

Classifying as set forth above provides a number of cell RNA sequences classified as tumor allele sequences, another number classified as normal allele sequences, and a third number classified as unknown alleles. These cell RNA sequences are all from one cell. In view of the classifying, the methods can comprise identifying the cell as a tumor cell, a healthy cell, or an unknown cell, based at least in part on the classifying of each of the plurality of cell RNA sequences.

In some embodiments, the cell can be identified as a tumor cell if it contains a threshold number of tumor allele sequences. The threshold can be one tumor allele sequence, two tumor allele sequences, three tumor allele sequences, four tumor allele sequences, five tumor allele sequences, six tumor allele sequences, seven tumor allele sequences, eight tumor allele sequences, nine tumor allele sequences, ten tumor allele sequences, eleven tumor allele sequences, twelve tumor allele sequences, thirteen tumor allele sequences, fourteen tumor allele sequences. fifteen tumor allele sequences, sixteen tumor allele sequences, seventeen tumor allele sequences, eighteen tumor allele sequences, nineteen tumor allele sequences, twenty tumor allele sequences, or more tumor allele sequences.

In some embodiments, the identifying the cell can comprise a Bayesian analysis of the number of classified tumor allele sequences and the number of classified normal allele sequences. The result of the Bayesian analysis is a probability that the cell is a tumor cell, a healthy cell, or an unknown cell. The specific implementation of the Bayesian analysis can vary: however, the following factors should be borne in mind.

A tumor cell expresses both tumor and normal alleles. Accordingly, for a tumor cell, 50% of alleles can be expected to be tumor alleles, and 50% as normal alleles, assuming the tumor alleles are not present in increased copy number. It may also be the case that a tumor cell can possess multiple mutant alleles at any particular variant. For example, a tumor cell can present 50% of a first tumor allele and 50% of a second tumor allele.

A normal cell is expected to express only normal alleles.

Some cell RNA sequences that are in fact normal allele sequences can be falsely identified as tumor allele sequences, and vice versa, at frequencies proportional to the background and contextual error rates. In view of this latter observation, in some embodiments, identifying the cell can be further based in part on the general or background error rate of the RNA sequencing. Alternatively or in addition, in some embodiments, the identifying the cell can be based in part on the sequence-specific or contextual error rate of the RNA sequencing.

Any particular variant i has a genotype Gᵢ, comprising two alleles, with “1” representing a tumor allele and “0” representing a healthy allele. For each Gᵢ, there is a probability sᵢ that a tumor cell does not carry mutation i (i.e., sᵢ is the probability the tumor cell presents a genotype Gᵢ of 0/0). The probability sᵢ can be empirically estimated as max {0.01, 1-2*VAFᵢ}, where VAFᵢ is the variant allele frequency at i.

Also for each Gᵢ, there is a probability tᵢ that a normal cell presents a genotype Gᵢ of 0/1. The probability t, can be empirically estimated as 1/log2 (SQᵢ), where SQ is the somatic quality of variant i.

For each UMI j, aⱼ is the observed allele and ϵj is the observed sequencing error.

In some embodiments, the Bayesian analysis can involve the computation of a probability that a tumor allele sequence or a normal allele sequence is present in a tumor cell or a healthy cell, in view of an estimated sequencing error rate ε (incorporating both background and contextual error rates for the cell RNA sequence of the allele), as follows for each allele sequence:

$$P ( normal allele \vert healthy cell ) = 1 - ε \\ P ( tumor allele \vert healthy cell ) = ε / 3 \\ P ( normal allele \vert healthy cell ) = 1 / 2 - ( ε / 3 )$$

$$P ( tumor allele \vert healthy cell ) = 1 / 2 - ( ε / 3 )$$

In some embodiments, a tumor cell can be identified as such if the Bayesian analysis gives a probability greater than any selected real number between 0 and 1. In some embodiments, the tumor cell can be identified as such if the Bayesian analysis gives a probability greater than or equal to 0.10, such as greater than or equal to 0.11, greater than or equal to 0.12, greater than or equal to 0.13, greater than or equal to 0.14, greater than or equal to 0.15, greater than or equal to 0.16, greater than or equal to 0.17, greater than or equal to 0.18, greater than or equal to 0.19, greater than or equal to 0.2, greater than or equal to 0.21, greater than or equal to 0.22, greater than or equal to 0.23, greater than or equal to 0.24, greater than or equal to 0.25, greater than or equal to 0.26, greater than or equal to 0.27, greater than or equal to 0.28, greater than or equal to 0.29, greater than or equal to 0.3, greater than or equal to 0.31, greater than or equal to 0.32, greater than or equal to 0.33, greater than or equal to 0.34, greater than or equal to 0.35. greater than or equal to 0.36, greater than or equal to 0.37, greater than or equal to 0.38, greater than or equal to 0.39, greater than or equal to 0.4, greater than or equal to 0.41, greater than or equal to 0.42, greater than or equal to 0.43, greater than or equal to 0.44, greater than or equal to 0.45, greater than or equal to 0.46, greater than or equal to 0.47, greater than or equal to 0.48, greater than or equal to 0.49, greater than or equal to 0.5, greater than or equal to 0.51, greater than or equal to 0.52, greater than or equal to 0.53, greater than or equal to 0.54, greater than or equal to 0.55, greater than or equal to 0.56, greater than or equal to 0.57, greater than or equal to 0.58, greater than or equal to 0.59, greater than or equal to 0.6, greater than or equal to 0.61, greater than or equal to 0.62, greater than or equal to 0.63, greater than or equal to 0.64, greater than or equal to 0.65, greater than or equal to 0.66, greater than or equal to 0.67. greater than or equal to 0.68, greater than or equal to 0.69, greater than or equal to 0.7, greater than or equal to 0.71, greater than or equal to 0.72, greater than or equal to 0.73, greater than or equal to 0.74, greater than or equal to 0.75. greater than or equal to 0.76, greater than or equal to 0.77, greater than or equal to 0.78, greater than or equal to 0.79, greater than or equal to 0.8, greater than or equal to 0.81, greater than or equal to 0.82, greater than or equal to 0.83, greater than or equal to 0.84, greater than or equal to 0.85, greater than or equal to 0.86, greater than or equal to 0.87, greater than or equal to 0.88, greater than or equal to 0.89, greater than or equal to 0.9, greater than or equal to 0.91. greater than or equal to 0.92, greater than or equal to 0.93, greater than or equal to 0.94, greater than or equal to 0.95, greater than or equal to 0.96, greater than or equal to 0.97, greater than or equal to 0.98, or greater than or equal to 0.99.

As should be apparent, although the description herein has referred to sequencing RNA from a cell and identifying that cell as a tumor or healthy cell, the method can be performed in parallel on multiple cells from a tumor, thereby providing information regarding the cell type populations within the tumor.

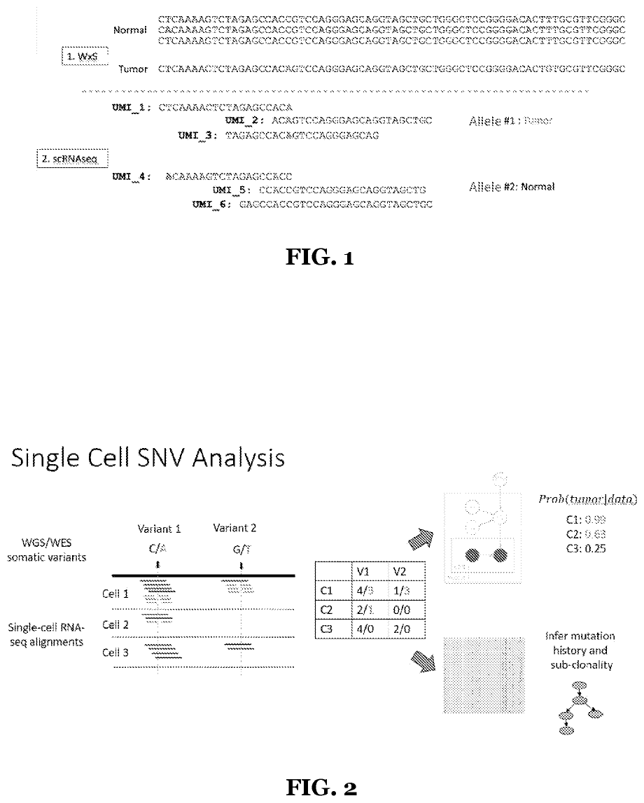

FIG. 2 depicts a simplified, hypothetical model in which multiple cell RNA sequences at two variant sites (Variant 1 and Variant 2) are classified as tumor allele sequences or healthy allele sequences in each of three cells (Cell 1, Cell 2, and Cell 3). In Cell 1, at Variant 1, four healthy (solid line) and three tumor (dashed line) allele sequences are identified, and at Variant 2, one healthy and three tumor allele sequences are identified. In Cell 2, at Variant 1, two healthy and one tumor allele sequences are identified, and at Variant 2, no allele sequences are identified. In Cell 3, at Variant 1, four healthy and zero tumor allele sequences are identified, and at Variant 2, two healthy and zero tumor allele sequences are identified. Applying the Bayesian analysis as set forth above, with ε=0.01 (corresponding to a phred quality score of 20), variant allele frequency (VAF)=0.25, and somatic quality (SQ)=30, yields the following probabilities that each cell is a tumor cell: Cell 1, 0.99; Cell 2, 0.63, Cell 3.0.25.

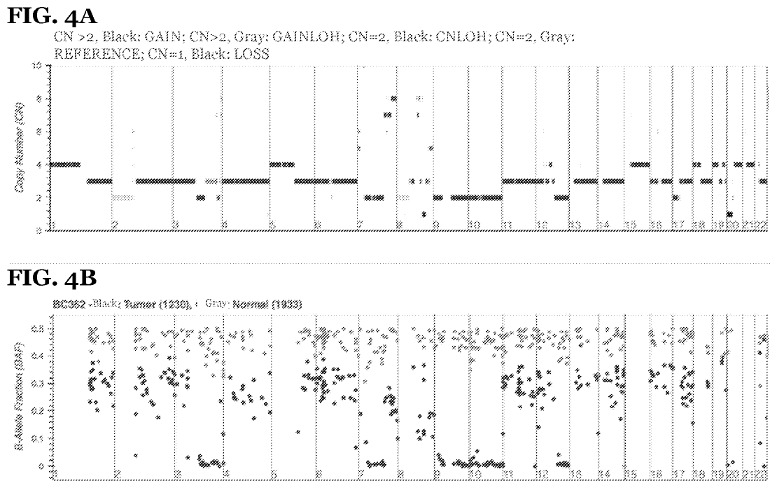

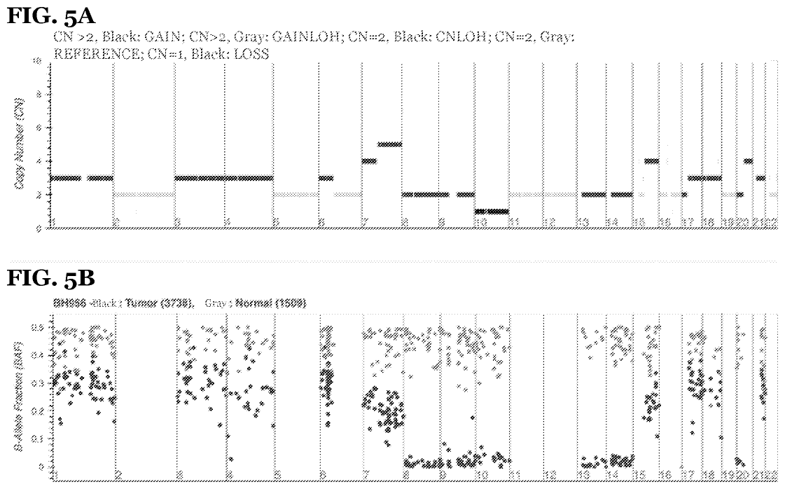

Germ-line variants are changes in DNA of a reproductive cell (e.g., sperm, egg) that become incorporated into every cell of the body of an offspring. Germ-line variations can be passed from parent to offspring (e.g., germ-line variants are hereditary). Germ-line variants can be present in both tumor and healthy cells. Nucleic acid sequences can contain multiple copies of a particular sequence (e.g., can have a copy number of greater than 1). Sequence regions in a nucleic acid sequence (e.g., a genome) can have any copy number. In many sequence regions, healthy cells typically have a copy number of two for a nucleotide sequence region within the genome (e.g., one allele for each chromosome in a pair of chromosomes). However, tumor cells can have an altered copy number (e.g., a copy number variation (CNV)) in comparison to healthy cells due to a mutation event in sections of the genome of the tumor cell. For example, FIGS. 4A and 5A show the copy number variation comparing healthy cells (e.g., copy number of two) to the cancer cells from two patient biopsies, BC362 and BH956, respectively. Copy number variation (CNV) in a cell can arise through any kind of mutation including but not limited to a single nucleotide polymorphism (SNP), an insertion, a deletion, a translocation, a duplication, or combinations thereof. Copy number and copy number variation can be determined through any type of nucleic acid sequencing including but not limited to whole genome sequencing and exome sequencing. When CNV occurs at a nucleic acid sequence region containing a germ-line variant (e.g., a region of heterozygosity in the DNA of a healthy cell), the allelic ratio can be altered. For example, a region of the genome in healthy cells can contain two alleles: allele 1 with a sequence of CATG, and allele 2 with a sequence of CATT. The healthy cell in this example has a copy number of two for this sequence region (e.g., one copy of allele 1 is on chromosome 2a and one copy of allele 2 is on chromosome 2b) resulting in an allelic ratio of 0.5 (e.g., half of the nucleic acids have a sequence of CATG and the other half have a sequence of CATT for these alleles). Continuing this example, if the healthy cell undergoes a duplication mutation of the region comprising allele 1 and becomes cancerous, the tumor cell would comprise two allele 1 sequences of CATG for each allele 2 sequence of CATT with a copy number of three for the region. The corresponding allelic ratio, represented as the B-allele frequency (e.g., the frequency of the minor allele), would decrease to about 0.33 (e.g., the minor allele would represent one third of the total alleles). As another example, a cancer cell could undergo deletion of the sequence comprising allele 1, resulting in a cancer cell only containing allele 2 with a copy number of one for the region and a B-allele frequency of 0 (e.g., only one allele exists with a sequence of CATT, loss of heterozygosity generating a hemizygous region). As another example, a cancer cell could undergo deletion of allele 1 and duplication of allele 2 (e.g., a copy-neutral loss of heterozygosity (CNLOH)), resulting in a cancer cell that contains only two copies of allele 2 and no copies of allele 1 with a copy number of two in the sequence region and a B-allele frequency of 0. In another example, a cancer cell could undergo two duplications of allele 1 and a deletion of allele 2, resulting in a cancer cell that contains three copies of allele 1 and no copies of allele 2 with a copy number of three in the region and a B-allele frequency of 0. FIGS. 4B and 5B show the calculated B-allele frequency of single cell RNA seq reads aligned to the genome for healthy (gray) and cancer (black) cells (BC362 and BH956 cancer cells, respectively) at germ-line variants for sequence regions of CNV.

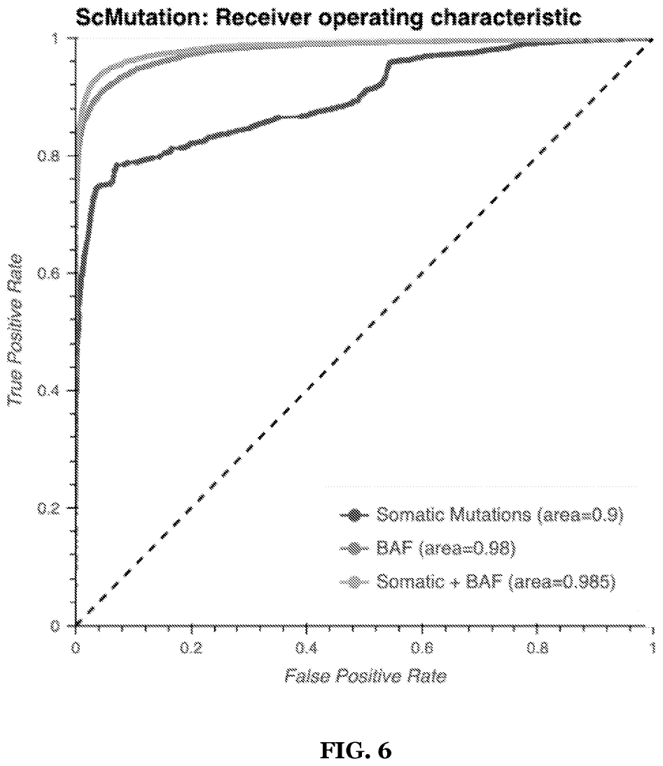

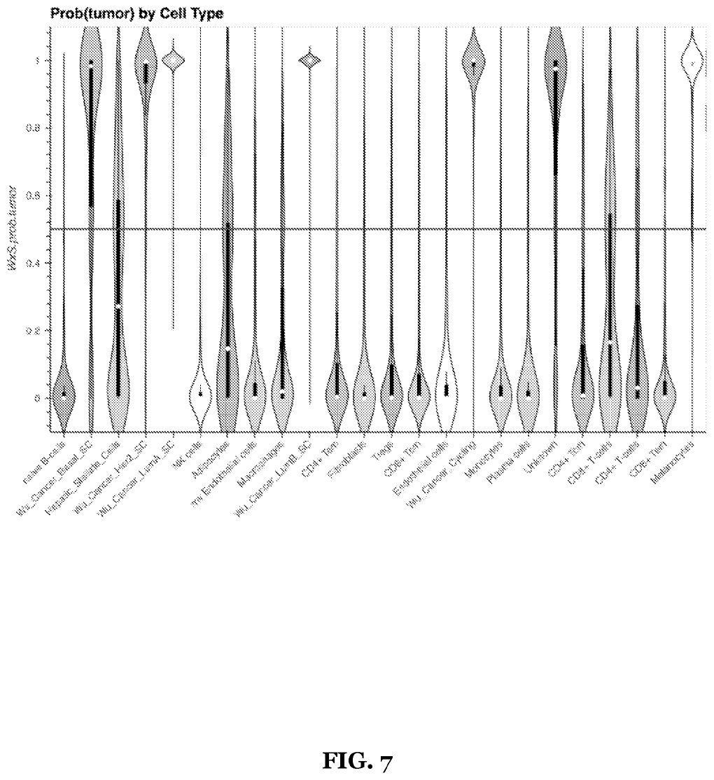

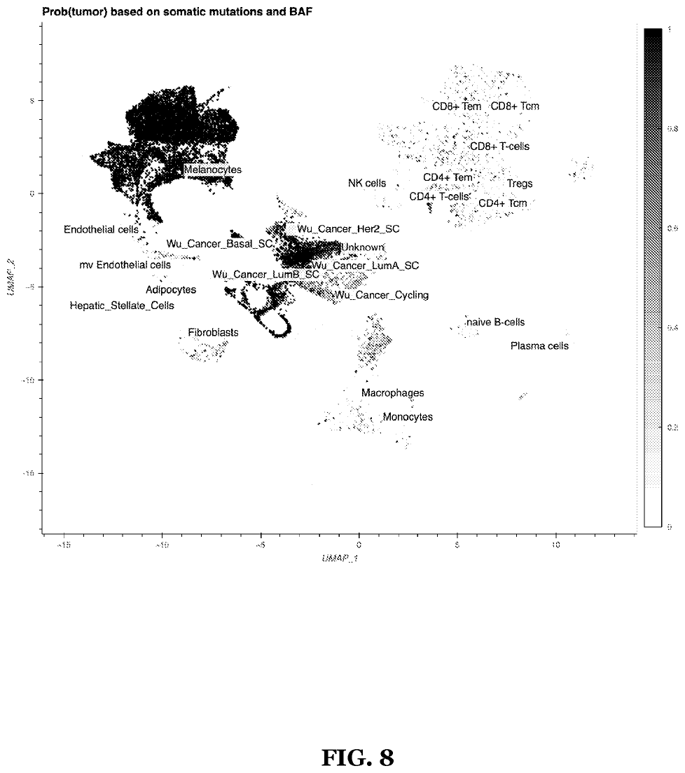

Methods of identification of cells (e.g., identification as tumor, healthy, or unknown cells; identification as a first cell, a second cell, or an unknown cell) can further be based at least in part on the allelic ratio of germ-line variants (e.g., heterozygous germ-line single nucleotide polymorphisms (SNPs), deletions, insertions, translocations, or combinations or hybrids thereof) in nucleotide sequences (e.g., RNA sequences, DNA sequences). Any method of comparing allelic ratios can be used. Methods of identifying healthy, tumor, and unknown cells can comprise sequencing DNA or RNA from a sample and generating a list of germ-line variants (e.g., a list of germ-line SNPs). A list of germ-line variants can be obtained from sequencing any nucleic acid including, but not limited to, bulk DNA (e.g., obtained by whole genome sequencing of bulk DNA, genomic DNA, cDNA obtained by reverse transcription), bulk RNA, single cell RNA (e.g., obtained by single RNA sequencing, single-nucleus RNA sequencing), single cell DNA (e.g., single cell whole-genome sequencing), or combinations thereof. Methods can comprise identifying germ-line variants in first and second sample bulk DNA sequences. Methods of identifying cells based at least in part on somatic mutations can be further improved in terms of higher true positive rate and lower false positive rate by including determination of B-allele frequency (BAF) of germ-line variants. An exemplary improvement in a method of identifying cells is shown in FIG. 6. which displays receiver operating characteristic (ROC) curves of multiple methods with or without somatic mutation and B-allele frequency determinations. Exemplary depiction of the identification of cells (e.g., patient isolates) by determining the probability a cell is a tumor cells is shown in FIG. 7 (as a graph of violin plots of cell type versus probability a cell is a tumor cell) and FIG. 8 (a graph of a single cell clustering analysis showing the probability each cell is a tumor cell). In some embodiments methods of identifying a cell as a first cell, a second cell, or an unknown cell, are based at least in part on B-allele frequency of germ-line variants in cell RNA sequences (e.g., single cell RNA sequences). In such embodiments, the method may further comprise identifying germ-line variants in the first sample bulk DNA sequences and second sample bulk DNA sequences.

Methods can further comprise determining copy number at any sequence region in a sample (e.g., a bulk DNA or RNA sample). Particularly suitable sequence regions for determining copy number can include sequence regions comprising a germ-line variant. In some embodiments, the methods comprise the step of determining a copy number for each sequence region comprising each germ line variant in a first sample bulk DNA sequence and a second sample bulk DNA sequence.

Methods can include the step of selecting one or more ‘determinative germ-line variants’ (DGLVs). The term ‘determinative germ-line variants’ as used herein refers to germ-line variants that 1) differ in B-allele frequency between a first sample and a second sample and/or 2) the copy number of a sequence region comprising the germ-line variant differs between the first sample and second sample. In methods of this disclosure, the B-allele frequency between two samples can be statistically different. The first B-allele frequency (e.g., the B-allele frequency of a germ-line variant in a first sample) and the second B-allele frequency (e.g., the B-allele frequency of a germ-line variant in a second sample) can be statistically different. The copy number of a sequence region comprising a germ-line variant can be expressed as a ratio of a copy number in the second sample and a copy number in the first sample (e.g., a copy number ratio). DGLVs selected from the germ-line variants can be encompassed by a sequence region that has a ratio of copy numbers that is not 1:1 (e.g., the copy number of the sequence region in the second sample is not equivalent to the copy number of the sequence region in the first sample). For example, the DGLV can be selected from a sequence region that is a duplication event (e.g., a sequence region wherein one allele of a pair alleles was duplicated, resulting in a copy number of three) in one of the samples. DGLVs selected from the germ-line variants can be encompassed by a sequence region that has a ratio of copy numbers that is 1:1 (e.g., the copy number of the sequence region in the second sample is equivalent to the copy number of the sequence region in the first sample). For example, the DGLV can be selected from a sequence region that is a copy neutral loss of heterozygosity (e.g., a region of deletion of one allele and duplication of the other allele) in one of the samples. In some embodiments, the method comprises the step of selecting one or more determinative germ-line variants (DGLVs) from the germ-line variants with a first B-allele frequency from the first sample bulk DNA sequence and a second B-allele frequency from the second sample bulk DNA sequence. In such embodiments, the first B-allele frequency and the second B-allele frequency can be statistically different. In such embodiments, the sequence region comprising each DGLV can have a ratio of the copy number in the second sample bulk DNA sequence to the copy number in the first sample bulk DNA sequence that is not 1:1. In some embodiments, the sequence region comprising each DGLV has a ratio of the copy number in the second sample bulk DNA sequence to the copy number in the first sample bulk DNA sequence that is 1:1. In some embodiments, the DGLVs differ both in B-allele frequency and copy number of the encompassing sequence region between a first sample and a second sample. In some embodiments, the DGLVs differ in only B-allele frequency and not in copy number between a second sample and a first sample.

Sequence regions of a nucleic acid sequence (e.g., a genome) can have any copy number. A sequence region (e.g., a sequence region comprising a DGLV) can have a copy number ranging from about 1 to about 20, e.g., about 1 to about 19, about 1 to about 18, about 1 to about 17, about 1 to about 16, about 1 to about 15, about 1 to about 14, about 1 to about 13, about 1 to about 12, about 1 to about 11, about 1 to about 10, about 1 to about 9, about 1 to about 8, about 1 to about 7, about 1 to about 6, about 1 to about 5, about 1 to about 4, about 1 to about 3, about 1 to about 2, about 2 to about 20, about 3 to about 20, about 4 to about 20, about 5 to about 20, about 6 to about 20, about 7 to about 20, about 8 to about 20, about 9 to about 20, about 10 to about 20, about 11 to about 20, about 12 to about 20, about 13 to about 20, about 14 to about 20. about 15 to about 20, about 16 to about 20, about 17 to about 20, about 18 to about 20, about 19 to about 20, about 2 to about 19, about 2 to about 18, about 2 to about 17, about 2 to about 16, about 2 to about 15, about 2 to about 14, about 2 to about 13, about 2 to about 12, about 2 to about 11, about 2 to about 10, about 2 to about 9, about 2 to about 8, about 2 to about 7, about 2 to about 6, about 2 to about 5, about 2 to about 4, about 2 to about 3, about 3 to about 19, about 3 to about 18, about 3 to about 17, about 3 to about 16, about 3 to about 15, about 3 to about 14, about 3 to about 13, about 3 to about 12, about 3 to about 11, about 3 to about 10, about 3 to about 9, about 3 to about 8, about 3 to about 7, about 3 to about 6, about 3 to about 5, about 3 to about 4, about 4 to about 19, about 4 to about 18, about 4 to about 17, about 4 to about 16, about 4 to about 15. about 4 to about 14, about 4 to about 13, about 4 to about 12, about 4 to about 11, about 4 to about 10, about 4 to about 9, about 4 to about 8, about 4 to about 7, about 4 to about 6, or about 4 to about 5. The sequence region can have a copy number of 1, 2, 3, 4, 5, 6, 7. 8, 9, 10, 11, 12, 13, 14, 15, 16, 17, 18, 19, 20 or more. In some embodiments, the sequence region has a copy number of 1. In some embodiments, the sequence region has a copy number of 2. In some embodiments, the sequence region has a copy number of 3. In some embodiments, the sequence region has a copy number of 4. In some embodiments, the sequence region has a copy number of 5. In some embodiments, the sequence region has a copy number of 6. In some embodiments, the sequence region has a copy number of 7. In some embodiments, the sequence region has a copy number of 8. In some embodiments, the sequence region has a copy number of 9. In some embodiments, the sequence region has a copy number of 10.

Sequence regions encompassing DGLVs can have a ratio of copy numbers of second sample to first sample of about 1:1, about 2:3, about 1:2, about 2:5, about 1:3, about 2:7, about 1:4, about 2:9, about 1:5, about 2:11, about 1:6, about 2:13, about 1:7, about 2:15, about 1:8, about 2:17, about 1:9, about 2:19, about 1:10, about 2:1, about 3:2, about 3:1, about 4:3. about 4:1. about 5:4. about 5:3. about 5:2. about 5:1. about 6:5. about 6:3. about 6:1. about 7:6, about 7:5, about 7:4, about 7:3, about 7:2, about 7:1, about 8:7, about 8:5, about 8:3, about 8:1, about 9:8, about 9:7, about 9:6, about 9:5, about 9:4, about 9:3, about 9:2. about 9:1, about 10:9, about 10:7, about 10:3, about 10:1, about 11:10, about 11:9, about 11:8, about 11:7, about 11:6, about 11:5, about 11:4, about 11:3, about 11:2, about 11:1, about 12:11, about 12:7, about 12:5, or about 12:1. In some embodiments, the sequence regions encompassing DGLVs can have a ratio of copy numbers of about 2:3, about 1:2, about 2:5, about 1:3, about 2:7, about 1:4, about 2:9, or about 1:5. In some embodiments, the sequence regions encompassing DGLVs can have a ratio of copy numbers of about 2:1. In some embodiments. the sequence regions encompassing DGLVs can have a ratio of copy numbers of about 1:1.

Statistical significance can be obtained by any statistical test including, but not limited to binomial test, Kruskal-Wallis one-way analysis of variance, Mann-Whitney U test, Siegel-Tukey test, student's T test, Tukey's range test, or a combination or hybrid thereof. In some embodiments, the statistical test is selected from the group consisting of binomial test, Kruskal-Wallis one-way analysis of variance, Mann-Whitney U test, Siegel-Tukey test, student's T test, Tukey's range test, and combinations and hybrids thereof.

The statistical difference as determined by a statistical test can be any difference including a difference with a probability under the assumption of no effect or no difference (e.g., null hypothesis) of obtaining a result equal to or more extreme than what is actually observed (p) of less than 0.1, e.g., less than 0.095, less than 0.090, less than 0.085, less than 0.080, less than 0.075, less than 0.070, less than 0.065, less than 0.060, less than 0.055, less than 0.050, less than 0.045. less than 0.040, less than 0.035. less than 0.030, less than 0.025, less than 0.020, less than 0.015, less than 0.010, less than 0.005, or less than 0.001, or less than 0.0001. In some embodiments, the statistical difference (p) is less than 0.050 and is determined by a statistical test. In some embodiments, the first B allele frequency from the first sample bulk DNA sequence and a second B allele frequency from a second sample bulk DNA sequence are statistically different and the statistical difference (p) is less than 0.05 and is determined by a statistical test. In some embodiments, the statistical test is a binomial test and the statistical difference (p) is less than 0.050.

Methods can further comprise aligning nucleic acid sequences with a germ-line variant (e.g., a DGLV). The nucleic acid sequences can be any type of nucleic acid including, but not limited to DNA (e.g., genomic DNA, cDNA, single cell DNA) or RNA (e.g., single cell RNA). In some embodiments, methods comprise the step of aligning each cell RNA sequence (e.g., each single cell RNA sequence) with each of the DGLVs. Any number of DGLVs can be selected from the germ-line variants and aligned to a nucleic acid sequence (e.g., a single cell RNA sequence). The number of DGLVs selected from the germ-line variants can range from about 1 to about 20,000, e.g., about 1 to about 2, about 1 to about 3, about 1 to about 4, about 1 to about 5, about 1 to about 6, about 1 to about 7, about 1 to about 8, about 1 to about 9, about 1 to about 10, about 1 to about 12, about 1 to about 14, about 1 to about 16, about 1 to about 18, about 1 to about 20, about 1 to about 22, about 1 to about 24, about 1 to about 26, about 1 to about 28, about 1 to about 30. about 1 to about 33, about 1 to about 36, about 1 to about 39, about 1 to about 42, about 1 to about 46, about 1 to about 50, about 1 to about 55, about 1 to about 60, about 1 to about 66, about 1 to about 72, about 1 to about 79, about 1 to about 87, about 1 to about 96, about 1 to about 100, about 1 to about 120, about 1 to about 140, about 1 to about 160, about 1 to about 180, about 1 to about 200, about 1 to about 250, about 1 to about 300, about I to about 350, about 1 to about 400, about 1 to about 450, about 1 to about 500, about 1 to about 550, about 1 to about 600, about 1 to about 650, about 1 to about 700, about 1 to about 750, about 1 to about 800, about 1 to about 850, about 1 to about 900, about 1 to about 950, about 1 to about 1000, about 1 to about 1100, about 1 to about 1200, about 1 to about 1300, about 1 to about 1400, about 1 to about 1500, about 1 to about 1600, about 1 to about 1700, about 1 to about 1800, about 1 to about 1900, about 1 to about 2000, about 1 to about 2200, about 1 to about 2400, about 1 to about 2600, about 1 to about 2800, about 1 to about 3000, about 1 to about 3300, about 1 to about 3600, about 1 to about 3900, about 1 to about 4300, about 1 to about 4700, about 1 to about 5000, about 1 to about 5500, about 1 to about 6000, about 1 to about 6500, about 1 to about 7000, about 1 to about 7500, about 1 to about 8000, about 1 to about 8500, about 1 to about 9000, about 1 to about 9500, about 1 to about 10,000, about 1 to about 11,000, about 1 to about 1 to about 12,000, about 1 to about 13,000, about 1 to about 14,000, about 1 to about 15,000, about 1 to about 16,000, about 1 to about 17,000, about 1 to about 18,000, about 1 to about 19,000, about 4 to about 20,000, about 5 to about 20,000, about 6 to about 20,000, about 7 to about 20,000, about 8 to about 20,000, about 9 to about 20,000, about 10 to about 20,000, about 15 to about 20,000, about 20 to about 20,000, about 25 to about 20.000, about 30 to about 20,000, about 35 to about 20,000, about 40 to about 20,000, about 45 to about 20,000, about 50 to about 20.000, about 60 to about 20.000, about 70 to about 20.000, about 80 to about 20,000, about 90 to about 20,000, about 100 to about 20,000, about 120 to about 20,000, about 140 to about 20,000, about 160 to about 20,000, about 180 to about 20,000, about 200 to about 20,000, about 240 to about 20,000, about 280 to about 20,000, about 320 to about 20,000, about 380 to about 20,000, about 440 to about 20,000, about 480 to about 20,000, about 560 to about 20,000, about 600 to about 20,000, about 650 to about 20,000, about 700 to about 20,000, about 800 to about 20,000, about 900 to about 20,000, about 1000 to about 20,000, about 1200 to about 20,000, about 1400 to about 20,000, about 1600 to about 20,000, about 1800 to about 20,000, about 2000 to about 20,000, about 2400 to about 20.000, about 2800 to about 20.000, about 3200 to about 20.000, about 3600 to about 20,000, about 4000 to about 20,000, about 4400 to about 20,000, about 4800 to about 20,000, about 5200 to about 20,000, about 5700 to about 20,000, about 6200 to about 20,000, about 7000 to about 20,000, about 8000 to about 20,000, about 9000 to about 20,000, about 10,000 to about 20,000, about 12,000 to about 20,000, about 14,000 to about 20,000, about 16,000 to about 20,000, about 18,000 to about 20,000, about 2 to about 20,000, about 3 to about 18,000, about 4 to about 16,000, about 5 to about 15,000, or about 6 to about 12,000.

Methods can further comprise determining an allele fraction or allelic frequency (e.g., a B allele frequency) of each germ-line variant (e.g., each DGLV) in the nucleic acids (e.g., single cell RNA sequences, single cell DNA sequences). In some embodiments, the methods comprise the step of determining the B-allele frequency of each DGLV in the cell RNA sequences.

Any germ-line variant can serve as the basis for determining B-allele frequency of a cell. Germ-line variants suitable for allelic ratio determination can include, but are not limited to, single nucleotide polymorphisms, insertions, deletions, translocations, or combinations thereof. Germ-line variants can result in any type of mutation in a protein gene product, including synonymous and non-synonymous mutations. In some embodiments, the germ-line variant is a mutation selected from the group consisting of a single nucleotide polymorphism, an insertion, a deletion, a translocation, and combinations thereof.

Cells from a first sample can have a CNV compared to cells from a second sample. Cells from a first sample with a CNV compared to cells from a second sample can have any B-allele frequency of germ-line variants (e.g., DGLVs). Cells with a CNV compared to healthy cells, such as cancer cells, can have any B-allele frequency of DGLVs. Cells with a CNV. such as cancer cells. can have a B-allele frequency of a germ-line variant (e.g., a DGLV) ranging from about 0.00 to about 0.5, e.g., about 0.00 to about 0.45, about 0.00 to about 0.42, about 0.00 to about 0.40, about 0.00 to about 0.38, about 0.00 to about 0.36, about 0.00 to about 0.34, about 0.00 to about 0.32, about 0.00 to about 0.30, about 0.00 to about 0.28, about 0.00 to about 0.26, about 0.00 to about 0.24, about 0.00 to about 0.22, about 0.00 to about 0.20, about 0.00 to about 0.19, about 0.00 to about 0.18, about 0.00 to about 0.17, about 0.00 to about 0.16, about 0.00 to about 0.15, about 0.00 to about 0.14, about 0.00 to about 0.13, about 0.00 to about 0.12, about 0.00 to about 0.11, about 0.00 to about 0.10, about 0.00 to about 0.09, about 0.00 to about 0.08, about 0.00 to about 0.07, about 0.00 to about 0.06, about 0.00 to about 0.05, about 0.00 to about 0.04, about 0.00 to about 0.03, about 0.00 to about 0.02, about 0.00 to about 0.01, about 0.05 to about 0.40, about 0.1 to about 0.40, about 0.15 to about 0.40, about 0.20 to about 0.40, about 0.25 to about 0.40, about 0.30 to about 0.40, about 0.35 to about 0.40, about 0.05 to about 0.37, about 0.10 to about 0.37, about 0.15 to about 0.37, about 0.20 to about 0.37, about 0.25 to about 0.37, about 0.30 to about 0.37, about 0.31 to about 0.35, about 0.29 to about 0.37. about 0.27 to about 0.39, about 0.23 to about 0.27, about 0.21 to about 0.29, about 0.19 to about 0.31, about 0.17 to about 0.33, about 0.15 to about 0.35, about 0.13 to about 0.37, about 0.12 to about 0.39, about 0.10 to about 0.41, about 0.18 to about 0.20, about 0.16 to about 0.22, about 0.14 to about 0.24, about 0.12 to about 0.26, about 0.05 to about 0.38, or about 0.10 to about 0.28. In some embodiments, the B-allele frequency of the DGLV in the cell RNA sequences validating a first cell range from about 0.00 to about 0.32.

Cells validated as second cells can have any B-allele frequency (e.g., a B-allele frequency of DGLV. a B-allele frequency of germ-line variants). Cells validated as second cells (e.g., healthy cells, non-tumor cells) can have a B-allele frequency (e.g., a B-allele frequency of DGLV, a B-allele frequency of germ-line variants ranging from about 0.00 to about 0.5, e.g., about 0.00 to about 0.45, about 0.00 to about 0.42, about 0.00 to about 0.40, about 0.00 to about 0.38, about 0.00 to about 0.36, about 0.00 to about 0.34, about 0.00 to about 0.32, about 0.00 to about 0.30, about 0.00 to about 0.28, about 0.00 to about 0.26, about 0.00 to about 0.24, about 0.00 to about 0.22, about 0.00 to about 0.20, about 0.00 to about 0.19, about 0.00 to about 0.18, about 0.00 to about 0.17. about 0.00 to about 0.16, about 0.00 to about 0.15, about 0.00 to about 0.14, about 0.00 to about 0.13, about 0.00 to about 0.12, about 0.00 to about 0.11, about 0.00 to about 0.10, about 0.00 to about 0.09, about 0.00 to about 0.08, about 0.00 to about 0.07, about 0.00 to about 0.06, about 0.00 to about 0.05, about 0.00 to about 0.04, about 0.00 to about 0.03, about 0.00 to about 0.02, about 0.00 to about 0.01, about 0.35 to about 0.50, about 0.37 to about 0.50, about 0.39 to about 0.50, about 0.40 to about 0.50, about 0.41 to about 0.50, about 0.42 to about 0.50, about 0.43 to about 0.50, about 0.44 to about 0.50, about 0.45 to about 0.50, about 0.46 to about 0.50, about 0.47 to about 0.50, about 0.48 to about 0.50, about 0.49 to about 0.50, about 0.23 to about 0.43, about 0.25 to about 0.41, about 0.27 to about 0.39, about 0.29 to about 0.37, about 0.31 to about 0.35, about 0.32 to about 0.34, about 0.15 to about 0.35, about 0.17 to about 0.33, about 0.19 to about 0.31, about 0.21 to about 0.29, about 0.23 to about 0.27, about 0.24 to about 0.26, about 0.10 to about 0.30. about 0.12 to about 0.28. about 0.14 to about 0.26, about 0.16 to about 0.24, about 0.18 to about 0.22, or about 0.19 to about 0.21. In some embodiments, the B-allele frequency of the DGLV in the cell sequences (single cell RNA sequences) validating a second cell range from about 0.40 to about 0.50.

A B-allele frequency can be not statistically different from any value. The B-allele frequency can be not statistically different from 0.50, 0.49, 0.48, 0.47, 0.46, 0.45, 0.44, 0.43, 0.42, 0.41, 0.40, 0.39, 0.38, 0.37, 0.36, 0.35, 0.34, 0.33, 0.32, 0.31, 0.30, 0.29, 0.28, 0.27, 0.26, 0.25, 0.33, 0.32, 0.31, 0.30, 0.29, 0.28, 0.27, 0.26, 0.25, 0.24, 0.23, 0.22, 0.21, 0.21, 0.20, 0.19, 0.18, 0.17, 0.16, 0.15, 0.143, 0.167, 0.14, 0.13, 0.125, 0.12, 0.111, 0.11, 0.10, 0.09, 0.08, 0.07, 0.06, 0.05, 0.04, 0.03, 0.02, or 0.01. In some embodiments, the B-allele frequency is not statistically different from 0.50. In some embodiments, the B-allele frequency is not statistically different from 0.33. In some embodiments, the B-allele frequency is not statistically different from 0.25. In some embodiments, the B-allele frequency is not statistically different from 0.20. In some embodiments. the B-allele frequency is not statistically different from 0.167. In some embodiments, the B-allele frequency is not statistically different from 0.143. In some embodiments, the B-allele frequency is not statistically different from 0.125. In some embodiments, the B-allele frequency is not statistically different from 0.111. In some embodiments, the B-allele frequency is not statistically different from 0.10. In some embodiments, cells with a B-allele frequency that is not significantly different from 0.50 are identified as healthy cells.

A B-allele frequency can be statistically different from any value. The B-allele frequency can be statistically different from 0.50, 0.49, 0.48, 0.47, 0.46, 0.45, 0.44, 0.43, 0.42, 0.41, 0.40, 0.39, 0.38, 0.37, 0.36, 0.35, 0.34, 0.33, 0.32, 0.31, 0.30, 0.29, 0.28, 0.27, 0.26, 0.25, 0.24, 0.23, 0.22, 0.21, 0.21, 0.20, 0.19, 0.18, 0.17, 0.167, 0.16, 0.15. 0.143, 0.14, 0.13, 0.125, 0.12, 0.111, 0.11, 0.10, 0.09, 0.08, 0.07, 0.06, 0.05, 0.04, 0.03, 0.02, or 0.01. In some embodiments, the B-allele frequency is statistically different from 0.50. In some embodiments, the B-allele frequency is statistically different from 0.33. In some embodiments, the B-allele frequency is statistically different from 0.25. In some embodiments, the B-allele frequency is statistically different from 0.20. In some embodiments, the B-allele frequency is statistically different from 0.167. In some embodiments, the B-allele frequency is statistically different from 0.143. In some embodiments, the B-allele frequency is statistically different from 0.125. In some embodiments, the B-allele frequency is statistically different from 0.111. In some embodiments, the B-allele frequency is statistically different from 0.10. In some embodiments, cells with a B-allele frequency that is significantly different from 0.50 are identified as tumor cells.

### F. Further Determinations Regarding Tumor Cells

The methods described herein can distinguish tumor cells from non-tumor cells. However, not all tumor cells are identical. The mutational processes that give rise to original tumor cells can lead to subclones with further mutations. The subclones can include mutations that allow them to survive therapies that are effective against their progenitors. Identifying subclones in a subject's tumor can provide the clinician with additional information to customize a treatment regimen for the subject.

In some embodiments, if the cell is identified as a tumor cell, the method can further comprise determining the subclone status of the tumor cell. In some embodiments. determining the subclone status can involve determining the co-occurrence of mutations at multiple alleles of a cell.

In some embodiments, if the cell is identified as a tumor cell, the method can further comprise determining the mutational history of the tumor cell. In some embodiments, determining the mutation history of the tumor cell can involve clustering variants based on their prevalence in all cells.

Returning to the simplified, hypothetical model of FIG. 2, the information presented regarding Variant 1 and Variant 2 for each of Cells 1-3 can be used to infer mutation history and subclonality.

The co-occurrence of variants in a single cell implies a history of clonal evolution using network inference techniques. For example, returning to FIG. 2, both Cell 1 and Cell 2 have Variant 1. However, only Cell 1 has both Variant 1 and Variant 2, and no tumor cells have only Variant 2. This makes it likely that there are two cancer subclones in the sample, one with only Variant 1 (Cell 2) and another with both Variant 1 and Variant 2 (Cell 1).

This also implies that the original cell that became cancerous had Variant 1 and that there has been no survival advantage for the tumor to remove Variant 1. The presence of Variant 2 in some but not all of the cells containing Variant 1 implies that Variant 2 arose after the tumor was established, resulting in a sub-clonal population of tumor cells containing both variants. Because no cells contain only Variant 2, it is very unlikely that the original cancerous cell contained Variant 2. Although it may be possible that the original cancerous cell contained both Variant 1 and Variant 2, and a subclone later lost Variant 2, this is unlikely because it would require two point mutations to occur at the same time, as opposed to only a single point mutation.

### G. Peptides Based on Tumor Sequences

The methods described herein can reveal mRNA sequences specific to tumor cells of a subject's tumor and not shared with the subject's normal cells, not even normal cells present in the tumor. Further, in some embodiments, the methods can reveal mRNA sequences specific to subclone tumor cells. The mRNA sequences specific to subclone tumor cells thus correspond to peptides expressed in the subclone tumor cells. These peptides can be used in the preparation of immunogenic compositions containing tumor-specific neoantigens, colloquially known as cancer vaccines. These immunogenic compositions can permit cancer therapy customized to the subject, taking into account one or more of the specific types of cancer, the status of the cancer, the immune status of the subject, and the MHC-type of the subject. In particular, by identifying one or more subclones of the tumor, the immunogenic composition can comprise peptides from all known subclones, thereby increasing the effectiveness of the immunogenic composition against all subclones and reducing the likelihood that one or more subclones can escape a subject's immune response and contribute to progression of the subject's tumor.

In some embodiments, the methods can further comprise generating at least one subclone peptide, each subclone peptide at least in part encoded by a cell RNA sequence identified as a tumor sequence and specific for the subclone status of the tumor cell. In some further embodiments, the methods can further comprise formulating an immunogenic composition comprising the at least one subclone peptide.

In some embodiments, the methods can further comprise generating at least one non-subclone peptide, each non-subclone peptide derived from a cell of a tumor of the subject which has a different subclone status than the tumor cell for which the subclone status was determined. In some further embodiments, the methods can further comprise including the at least one non-subclone peptide in the immunogenic composition.

The immunogenic composition can comprise at least about 1, about 2 about 3,about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50 or more tumor-specific neoantigen peptides.

The immunogenic composition can comprise up to about 100 tumor-specific neoantigens. The immunogenic composition can contain about 10-20 tumor-specific neoantigens, about 10-30 tumor-specific neoantigens, about 10-40 tumor-specific neoantigens, about 10-50 tumor-specific neoantigens, about 10-60 tumor-specific neoantigens, about 10-70 tumor-specific neoantigens, about 10-80 tumor-specific neoantigens, about 10-90 tumor-specific neoantigens, or about 10-100 tumor-specific neoantigens. Typically. the immunogenic composition comprises at least about 10 tumor-specific neoantigens. The immunogenic composition disclosed herein preferably comprises 10 to about 20 tumor-specific neoantigens. For example, the immunogenic composition can comprise about 10, about 11, about 12, about 13, about 14, about 15, about 16, about 17, about 18, about 19, or about 20 tumor-specific neoantigens. Preferably, the immunogenic composition can comprise about 19 tumor-specific neoantigens. Preferably, the immunogenic composition can comprise about 20 tumor-specific neoantigens. Each of the tumor-specific neoantigens in the immunogenic composition are preferably different.

The tumor-specific neoantigen peptides can be long peptides (peptides about 15 amino acid to about 30 amino acid in length) and/or short peptides (peptides about 5 amino acid to about 15 amino acid in length). Tumor-specific neoantigen long peptides are internalized by antigen-presenting cells and processed for MCH presentation. MHC class II molecules typically bind to peptides that are longer in length. MHC class II can accommodate peptides which are generally about 13 amino acids in length to about 25 amino acids in length. In embodiments, the one or more tumor-specific neoantigens are long peptides about 13 to 25 amino acids in length. MHC class I molecules typically bind to short peptides. Tumor-specific neoantigen short peptides bind directly to MHC molecules. MHC class I molecules can bind to short peptides. MHC class I molecules can accommodate peptides generally about 8 amino acids to about 10 amino acids in length.

One or more of the tumor-specific neoantigen peptides included in the immunogenic composition can be identified by the present methods.

The immunogenic composition can also comprise one or more of a helper peptide, an adjuvant, or a tumor-specific frameshift peptide.

### H. Methods of Treating Cancer

In some embodiments, wherein the methods comprise generating a subclone peptide and formulating an immunogenic composition comprising the subclone peptide, the methods can further comprise administering the immunogenic composition to the subject. By doing so, the subject's cancer can be treated.

The cancer can be any solid tumor or any hematological tumor. The tumor can be a primary tumor (e.g., a tumor that is at the original site where the tumor first arose). Solid tumors can include, but are not limited to, breast cancer tumors, ovarian cancer tumors, prostate cancer tumors, lung cancer tumors, kidney cancer tumors, gastric cancer tumors. testicular cancer tumors, head and neck cancer tumors, pancreatic cancer tumors, brain cancer tumors, and melanoma tumors. Hematological tumors can include, but are not limited to, tumors from lymphomas (e.g., B cell lymphomas) and leukemias (e.g., acute myelogenous leukemia, chronic myelogenous leukemia, chronic lymphocytic leukemia, and T cell lymphocytic leukemia).

The methods disclosed herein can be used for any suitable cancerous tumor, including hematological malignancy, solid tumors, sarcomas, carcinomas, and other solid and non-solid tumors. Illustrative suitable cancers include, for example, acute lymphoblastic leukemia (ALL), acute myeloid leukemia (AML), adrenocortical carcinoma, anal cancer, appendiceal cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer. macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer. oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor. Preferably, the cancer is melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, T-cell lymphocytic leukemia, bladder cancer, or lung cancer. Melanoma is of particular interest. Breast cancer, lung cancer, and bladder cancer are also of particular interest.

Immunogenic compositions stimulate a subject's immune system, especially the response of specific CD8+ T cells or CD4+ T cells. Interferon gamma produced by CD8+ and T helper CD4+ cells regulate the expression of PD-L1. PD-L1 expression in tumor cells is upregulated when attacked by T cells. Therefore, tumor vaccines may induce the production of specific T cells and simultaneously upregulate the expression of PD-L1, which may limit the efficacy of the immunogenic composition. In addition, while the immune system is activated, the expression of T cell surface reporter CTLA-4 is correspondingly increased, which binds with the ligand B7-1/B7-2 on antigen-presenting cells and plays an immunosuppressant effect. Thus, in some instances, the subject may further be administered an anti-immunosuppressive or immunostimulatory, such as a checkpoint inhibitor. Checkpoint inhibitors can include, but are not limited to, anti-CTL4-A antibodies, anti-PD-1 antibodies and anti-PD-L1 antibodies, inhibitors of the Lag3 pathway, the Tim3 pathway, the ICOS pathway, the OX-40 pathway. the GITR pathway, or the 4-1BB pathway. These checkpoint inhibitors bind to the immune checkpoint proteins of T cells to remove the inhibition of T cell function by tumor cells. Blockade of CTLA-4 or PD-L1 by antibodies can enhance the immune response to cancerous cells in the patient. CTLA-4 has been shown effective when following a vaccination protocol.

The immunogenic composition described herein can be administered to a subject that has been diagnosed with cancer, is already suffering from cancer, has recurrent cancer (i.e., relapse), or is at risk of developing cancer. The immunogenic composition described herein can be administered to a subject that is resistant to other forms of cancer treatment (e.g., chemotherapy, immunotherapy, or radiation). The immunogenic composition described herein can be administered to the subject prior to, in conjunctions, or after other standard of care cancer therapies (e.g., surgery, chemotherapy, immunotherapy, or radiation). The immunogenic composition described herein can be administered to the subject concurrently, after, or in combination to other standard of care cancer therapies (e.g., chemotherapy, immunotherapy, or radiation).

The subject can be a human, dog, cat, horse, or any animal for which a tumor specific response is desired.

The immunogenic composition described herein can be administered to the subject alone or in combination with other therapeutic agents. The therapeutic agent can be, for example, a chemotherapeutic agent, hormone-modulators, signaling cascade inhibitors, radiation, or immunotherapy. Any suitable therapeutic treatment for a particular cancer can be administered. Exemplary chemotherapeutic agents include, but are not limited to aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol®), pilocarpine, prochlorperazine, rituximab, tamoxifen, taxol, topotecan hydrochloride, trastuzumab, vinblastine, vincristine and vinorelbine tartrate. The subject may be administered a small molecule, or targeted therapy (e.g., kinase inhibitor). The subject may be further administered an anti-CTLA antibody or anti-PD-1 antibody or anti-PD-L1 antibody. Blockade of CTLA-4 or PD-L1 by antibodies can enhance the immune response to cancerous cells in the patient.

In some embodiments, the immunogenic composition can be administered prior to or simultaneously with delivering one or more other therapeutic agents for the tumor to the subject.

In some embodiments, one or more of the generating at least one subclone peptide, the formulating, the generating at least one non-subclone peptide (if performed as part of the method), the including the at least one non-subclone peptide (if performed as part of the method), and the administering of the immunogenic composition formulated in accordance with the methods disclosed herein can be performed after delivering one or more other therapeutic agents and/or another immunogenic composition to the subject.

### 5. EQUIVALENTS

It will be readily apparent to those skilled in the art that other suitable modifications and adaptions of the methods of the invention described herein are obvious and may be made using suitable equivalents without departing from the scope of the disclosure or the embodiments. Having now described certain compositions and methods in detail, the same will be more clearly understood by reference to the following examples, which are introduced for illustration only and not intended to be limiting.

### 6. EXAMPLES

The following are examples of methods and compositions of the invention. It is understood that various other embodiments may be practiced, given the general description provided herein.

### A. Example 1. Estimation of Percentage of Somatic Variants Detectable by scRNA Sequencing

As discussed herein, scRNA sequencing is generally limited to about 100 nucleotides from the 3′ end of an mRNA. This means that scRNA sequencing cannot provide information regarding the entirety of any transcript.

To determine the extent to which variants found by comparison of WxS sequences from healthy and tumor tissue can be mapped to scRNA sequencing reads, and the extent to which cells containing classifiable scRNA sequencing reads can be identified as tumor cells, five databases containing bulk DNA sequence data from healthy tissue and from tumor tissue, and scRNA sequence data from the corresponding tumor tissues were examined. In each database, somatic variants found by comparison between the bulk DNA sequence data from healthy and tumor tissue were mapped to scRNA sequencing reads. The number of cells containing classifiable scRNA sequencing reads was determined, as was the number of cells containing at least one scRNA sequencing read classified as a tumor allele sequence. The results are presented below in Table 1.

The results demonstrate that, in four out of five databases, about 10-25% of somatic variants indicative of tumors can be mapped to scRNA sequencing reads. In all five databases, about 10-40% of cells were found to contain at least one tumor allele sequence.

TABLE 1

| | Variants Mapped to | | Cells Containing Tumor Allele | |
|---|---|---|---|---|
| Database ID | scRNA data/Total | | Sequences/Total Cells Containing | |
| Number | Variants | | Classifiable Allele Sequences | |
| 1 | 63/605 | (10.4%) | 636/2535 | (25.1%) |
| 2 | 132/617 | (21.4%) | 2571/8104 | (31.7%) |
| 3 | 61/305 | (20.0%) | 1881/17538 | (10.7%) |
| 4 | 3584/182179 | (2.0%) | 7097/29103 | (24.4%) |
| 5 | 44/180 | (24.4%) | 931/2303 | (40.4%) |

### B. Example 2. Identification of Cells as Tumor Cells, Normal Cells, or Unknown Cells

From a tumor sample of a subject, 24 cells were subjected to scRNA sequencing. Reads from the scRNA sequencing were aligned with bulk DNA sequences from the subject's tumor and healthy tissue. The number of UMIs (unique reads) classified as tumor allele sequences, normal allele sequences, or unknown sequences for each cell were counted and are presented below in Table 2.

The probability that each cell was a tumor cell or a healthy cell was calculated from the total number of tumor allele sequences and healthy allele sequences, according to the following Bayesian formulation:

$$P ( normal allele \vert healthy cell ) = 1 - ε \\ P ( tumor allele \vert healthy cell ) = ε / 3 \\ P ( normal allele \vert healthy cell ) = 1 / 2 - ( ε / 3 )$$

$$P ( tumor allele \vert healthy cell ) = 1 / 2 - ( ε / 3 )$$

wherein ε is the sequencing error for each UMI, containing terms for background error rate, defined as the average of sequencing error rate for each read sharing the same UMI, followed by the correction of contextual errors where applicable.

Table 2 also presents the probability that each cell is a tumor cell. Values shown as “1” represent probabilities greater than or equal to 0.99995.

TABLE 2

| | | | Number of | Probability |
|---|---|---|---|---|
| Cell_ID | Number of | Number of | unknown | cell is a |
| Number | tumor UMIs | normal UMIs | UMIs | tumor cell |
| 1 | 0 | 1 | 0 | 0.4495 |
| 2 | 19 | 30 | 0 | 1 |
| 3 | 0 | 25 | 0 | 0.0297 |
| 4 | 0 | 10 | 0 | 0.1611 |
| 5 | 1 | 0 | 0 | 0.7035 |
| 6 | 3 | 0 | 0 | 0.9818 |
| 7 | 0 | 1 | 0 | 0.4739 |
| 8 | 0 | 7 | 0 | 0.2591 |
| 9 | 0 | 1 | 0 | 0.462 |
| 10 | 0 | 1 | 0 | 0.4739 |
| 11 | 0 | 1 | 0 | 0.348 |
| 12 | 0 | 1 | 0 | 0.4739 |
| 13 | 0 | 31 | 0 | 0.0097 |
| 14 | 0 | 2 | 0 | 0.3999 |
| 15 | 0 | 19 | 0 | 0.038 |
| 16 | 15 | 36 | 0 | 0.9999 |
| 17 | 1 | 1 | 0 | 0.7122 |
| 18 | 1 | 2 | 0 | 0.6127 |
| 19 | 1 | 2 | 0 | 0.7489 |
| 20 | 0 | 1 | 0 | 0.4739 |
| 21 | 0 | 13 | 0 | 0.1452 |
| 22 | 17 | 33 | 0 | 1 |
| 23 | 1 | 12 | 0 | 0.3598 |
| 24 | 1 | 16 | 0 | 0.1513 |

### C. Example 3. Comparison with Tumor Cell Determination by Gene Expression Profiling

To validate the identification of cells by the methods described herein, a heterogenous cell population comprising myeloid cells, natural killer (NK)/T cells, erythrocytes, fibroblasts, B cells, granulocytes, and melanoma cells was used. Cell types were determined based on gene expression profiles. Cells were also identified according to the methods described herein.

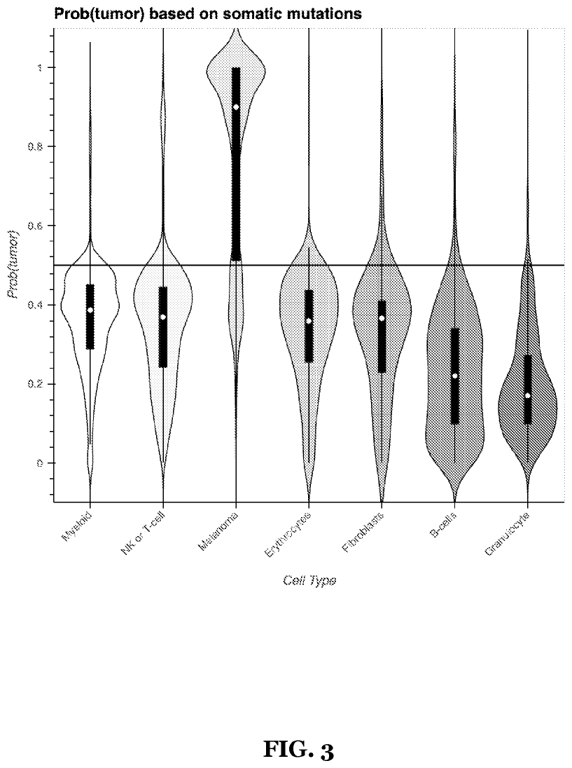

FIG. 3 shows that the majority of myeloid cells, natural killer (NK)/T cells, erythrocytes, fibroblasts, B cells, and granulocytes had less than a 0.5 (or 50%) probability of being tumor cells, whereas the vast majority of melanoma cells had a high probability of being tumor cells. This indicates the methods described herein yield per-cell tumor probabilities consistent with tumor cell identification by gene expression profiling.

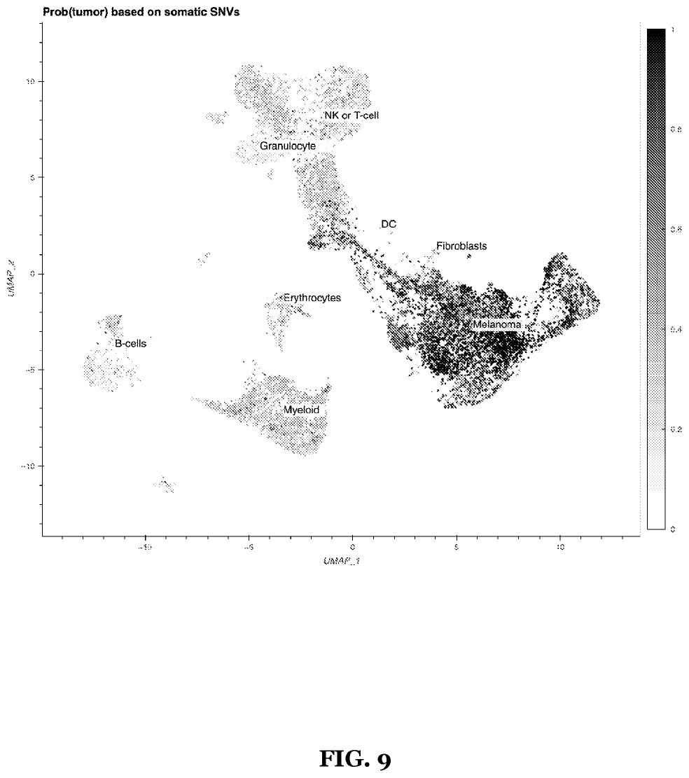

FIG. 9 shows the same cell populations as a graph of cell clustering analysis of single cell RNA sequencing results with the probability of each cell being a tumor cell shown in a gradient. These data show that most of the cells with a high probability of being a tumor cell are melanoma cells.

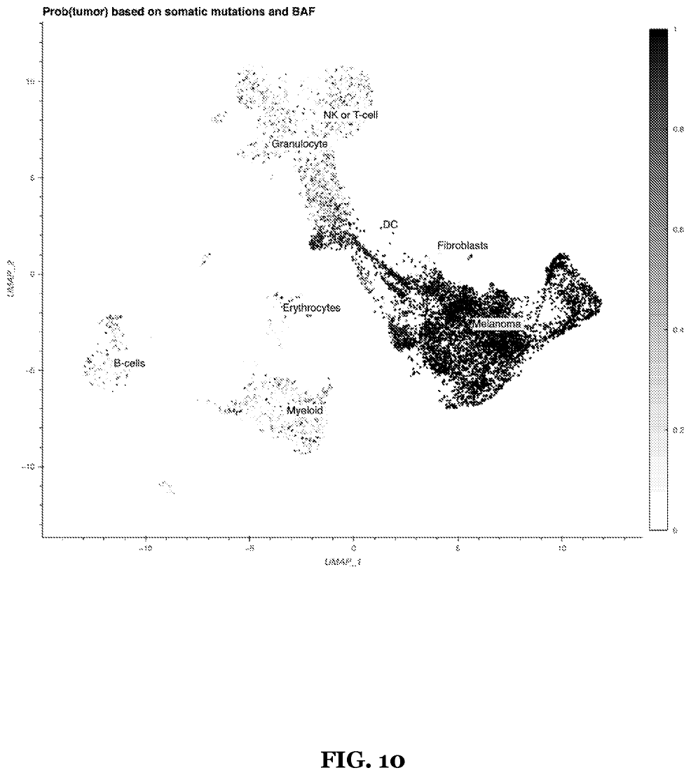

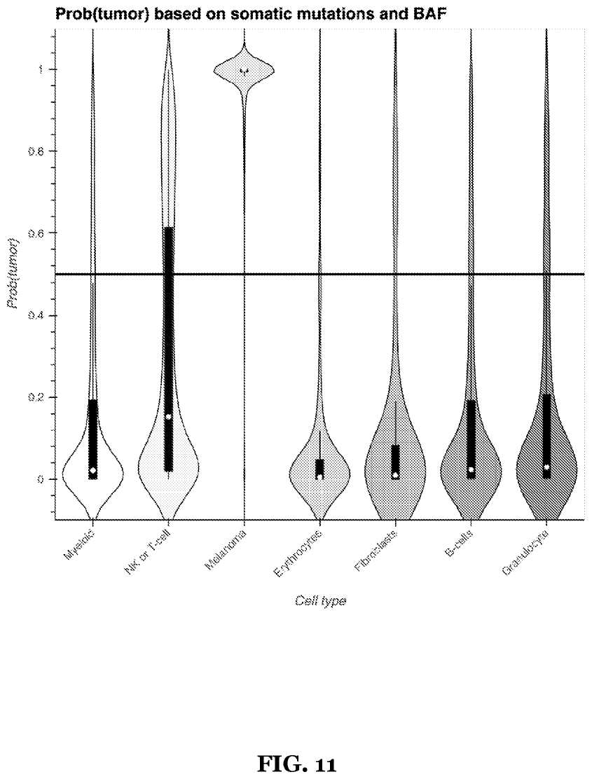

The identification method was further modified by validation of cell identifications by DGLV B-allele frequency as described herein. FIG. 11 shows that with B-allele frequency determination, the probability of correctly identifying melanoma cells as tumor cells (e.g., true positive rate), is increased to about 1.0 (e.g., about 100% probability), while the probability of identifying a healthy cell (e.g., a myeloid cell, a fibroblast cell) as a tumor cell (e.g., false positive rate) is decreased compared to the results of a method not based on B-allele frequency as shown in FIG. 3. This data is further depicted in FIG. 10 as a single cell RNA sequencing (scRNAseq) clustering analysis graph, which shows the probability that each cell is a tumor cell indicated by a gradient. The same depiction of a method that is based on somatic mutations but not based on B-allele frequency is shown in FIG. 9. As clearly seen by comparing FIG. 9 and FIG. 10, methods of cell identification have a higher probability of melanoma cells identified as cancer cells (e.g., a higher true positive rate) when based on B-allele frequency and somatic mutations. This improvement in achieving a true positive rate is further shown in FIG. 6 as an ROC curve comparing methods of cell identification disclosed herein.

D. Example 4. In Vitro B-Allele Frequency of Determinative Germ-Line Variants (DGLVs) in Healthy and Tumor Cells

The copy number of sequence regions of the BC362 and BH956 cancer cell genomes were determined computationally based on whole genome sequencing. BC362 and BH956 cancer cells showed a different pattern of copy number variation compared to healthy cells as shown in FIGS. 4A and 5A, respectively. Both genomes showed sequence regions comprising 1) duplication events with a higher copy number than 2. 2) deletion events with a copy number of 1, 3) copy neutral loss of heterozygosity (e.g., arising from a loss of one allele and one duplication of the remaining allele), 4) duplication events with loss of heterozygosity (e.g., arising from deletion of one allele and multiple duplications of the remaining allele), and 5) reference regions that show no change in copy number compared to the healthy cell genome. Healthy and cancer cells were analyzed by single cell RNA sequencing (scRNAseq) and the resulting sequences were aligned with germ-line variants in the genome. Germ-line variants contained within sequence regions of copy number variation in BC362 and BH956 were on average lower in B-allele frequency than in healthy cells, as shown in FIGS. 4B and 5B, respectively. Germ-line variants within sequence regions of lower copy number (a copy number of 1) or a loss of heterozygosity compared to healthy cells had a B-allele frequency of about 0). Germ-line variants within sequence regions with a higher copy number (a copy number of three or greater), had a B-allele copy number of less than 0.50 (e.g., about 0.05 to about 0.38). Healthy cells or reference regions with a copy number of two had a B-allele copy number of about 0.5 (e.g., about 0.40 to about 0.50).

### E. Example 5. Identification of Cells From Patient Samples

A diverse selection of cells from different patient sample cell types (24 cell types) were identified by methods disclosed herein based, at least in part, on both somatic mutations and germ-line variants. The results are shown in FIG. 7, which shows that multiple cancer cell types (e.g., basal-like breast cancer, Her2 enriched breast cancer) show a higher probability of tumor cell identification (true positive identification) compared to most non-tumor cell types (e.g., Tregs, fibroblasts, CD4+ T effector memory cells). These data are further depicted in FIG. 8.

What is claimed:

1. A method for classifying a cell present in a first sample from a subject, comprising:
sequencing first sample bulk DNA from the first sample from the subject;
sequencing second sample bulk DNA from a second sample from the subject;
classifying each somatic variant between the first sample bulk DNA sequence and the second sample bulk DNA sequence as a first sample allele if present in the first sample bulk DNA sequence or a second sample allele if present in the second sample bulk DNA sequence;
sequencing RNA from the cell, to yield a plurality of cell RNA sequences;
aligning each cell RNA sequence of the plurality of cell RNA sequences with the first sample bulk DNA sequence and the second sample bulk DNA sequence;
classifying each cell RNA sequence of the plurality of cell RNA sequences as a second allele sequence if the cell RNA sequence substantially aligns with a second sample allele from the second sample bulk DNA sequence, as a first allele sequence if the cell RNA sequence substantially aligns with a first sample allele from the first sample bulk DNA sequence, or as an unknown allele sequence if the cell RNA sequence does not substantially align with either the second sample bulk DNA sequence or the first sample bulk DNA sequence; and
identifying the cell as a first cell, a second cell, or an unknown cell, based at least in part on the classifying of each cell RNA sequence of the plurality of cell RNA sequences.

2. The method of claim 1, wherein the first sample is from a tumor and the second sample is from healthy tissue.

3. The method of claim 1, wherein the sequencing bulk DNA from the first sample or the sequencing bulk DNA from the second sample comprises whole genome sequencing or exome sequencing.

4. (canceled)

5. The method of claim 1, wherein the sequencing RNA from the cell yields a plurality of cell RNA sequences each comprising a unique molecular identifier (UMI) and about 100 nucleotides from the 3′ end of an RNA present in the cell.

6. The method of claim 1, further comprising determining a general error rate for the sequencing RNA from the cell or a sequence-specific error rate for each cell RNA sequence in the plurality of cell RNA sequences;
wherein the classifying each cell RNA sequence is based in part on the general error rate, the identifying the cell is further based in part on the general error rate, or the classifying each cell RNA sequence is based in part on the sequence-specific error rate.

7. (canceled)

8. The method of claim 1, wherein the identifying the cell comprises a Bayesian analysis of a number of first allele sequences and a number of second allele sequences.

9. The method of claim 2, wherein the first cell is a tumor cell and wherein the method further comprises the step of
determining a subclone status or the mutational history of the tumor cell.

10. (canceled)

11. The method of claim 9, further comprising generating a subclone peptide that is at least in part encoded by a cell RNA sequence from the tumor cell and specific for the subclone status of the tumor cell; and
formulating an immunogenic composition comprising the subclone peptide.

12. The method of claim 11, further comprising generating a non-subclone peptide, wherein the non-subclone peptide is derived from a cell that has a different subclone status than the tumor cell, wherein the cell that has a different subclone status from the tumor cell is from the tumor of the subject; and
including the non-subclone peptide in the immunogenic composition.

13. (canceled)

14. The method of claim 11, further comprising administering the immunogenic composition to the subject.

15. The method of claim 14, wherein the administering is performed prior to or simultaneously with delivering one or more other therapeutic agents for the tumor to the subject.

16. The method of claim 14, wherein one or more of the generating the subclone peptide, the formulating, and the administering are performed after delivering one or more other therapeutic agents and/or other immunogenic compositions to the subject.

17. (canceled)

18. The method of claim 1, further comprising the step of
validating the step of identifying the cell as a first cell, a second cell, or an unknown cell, based at least in part on an allelic frequency of germ-line variants in the cell RNA sequences.

19. The method of claim 1, further comprising the steps of
identifying germ-line variants in the first and the second sample bulk DNA sequences and determining a copy number at each sequence region comprising each germ-line variant in the first sample bulk DNA sequence and the second sample bulk DNA sequence;
selecting one or more determinative germ-line variants (DGLVs) from the germ-line variants with a first B-allele frequency from the first sample bulk DNA sequence and a second B-allele frequency from the second sample bulk DNA sequence, wherein the first B-allele frequency and the second B-allele frequency are statistically different, wherein the sequence region comprising each DGL V has a ratio of the copy number in the second sample bulk DNA sequence to the copy number in the first sample bulk DNA sequence;
aligning each cell RNA sequence of the plurality of cell RNA sequences with each of the DGLVs and determining a B-allele frequency of each DGLV in the plurality of cell RNA sequences; and
validating the step of identifying the cell as a first cell, a second cell, or an unknown cell, based at least in part on the B-allele frequency of each DGLV in the plurality of cell RNA sequences.

20. (canceled)

21. (canceled)

22. The method of claim 19, wherein the statistical difference is determined by a test selected from the group consisting of binomial test, Kruskal-Wallis one-way analysis of variance, Mann-Whitney U test, Siegel-Tukey test, student's T test, Tukey's range test, and combinations and hybrids thereof.

23. The method of claim 19, wherein the ratio of the copy numbers is about 2:3, about 2:1, about 1:1, about 1:2, about 2:5, about 1:3, about 2:7, about 1:4, about 2:9, or about 1:5.

24. (canceled)

25. (canceled)

26. The method of claim 19, wherein the first B-allele frequency is statistically different from 0.50 as determined by a first statistical test; or wherein the second B-allele frequency is not statistically different from 0.50 as determined by a second statistical test.

27. (canceled)

28. The method of claim 26, wherein the first statistical test and/or the second statistical test is a binomial test with p<0.050.

29. The method of claim 19, wherein the B-allele frequency of the DGLV in the plurality of cell RNA sequences validating the step of identifying the cell as a second cell ranges from about 0.40 to about 0.50.

30. The method of claim 19, wherein the B-allele frequency of the DGLV in the plurality of cell RNA sequences validating the step of identifying the cell as a first cell ranges from about 0.00 to about 0.32.


SEQUENCE LISTING (9 sequences)

SEQ ID NO: 1 - DNA, 70 bp, Homo sapiens
  source 1..70: mol_type = genomic DNA
ctcaaaagtctagagccaccgtccagggagcaggtagctgctgggctccggggacactttgcgttcgggc

SEQ ID NO: 2 - DNA, 70 bp, Homo sapiens
  source 1..70: mol_type = genomic DNA
cacaaaagtctagagccaccgtccagggagcaggtagctgctgggctccggggacactttgcgttcgggc

SEQ ID NO: 3 - DNA, 70 bp, Homo sapiens
  source 1..70: mol_type = genomic DNA
ctcaaaactctagagccacagtccagggagcaggtagctgctgggctccggggacactgtgcgttcgggc

SEQ ID NO: 4 - RNA, 20 nt, synthetic construct
  source 1..20: mol_type = other RNA
ctcaaaactctagagccaca

SEQ ID NO: 5 - RNA, 24 nt, synthetic construct
  source 1..24: mol_type = other RNA
acagtccagggagcaggtagctgc

SEQ ID NO: 6 - RNA, 23 nt, synthetic construct
  source 1..23: mol_type = other RNA
tagagccacagtccagggagcag

SEQ ID NO: 7 - RNA, 19 nt, synthetic construct
  source 1..19: mol_type = other RNA
acaaaagtctagagccacc

SEQ ID NO: 8 - RNA, 25 nt, synthetic construct
  source 1..25: mol_type = other RNA
ccaccgtccagggagcaggtagctg

SEQ ID NO: 9 - RNA, 29 nt, synthetic construct
  source 1..29: mol_type = other RNA
gagccaccgtccagggagcaggtagctgc